(19) United States
(12) Patent Application Publication
(10) Pub. No.: US 2015/0302157 A1
(43) Pub. Date: Oct. 22, 2015

(54) Apparatus, Method, and System for Counting Packaged, Consumable, Medical Items Such as Surgical Suture Cartridges

(71) Applicants: Ryan Mitchell Collar, Terrace Park, OH (US); Derek Kofi O. Boahene, Forest Hill, MD (US)

(72) Inventors: Ryan Mitchell Collar, Terrace Park, OH (US); Derek Kofi O. Boahene, Forest Hill, MD (US)

(21) Appl. No.: 14/255,026

(22) Filed: Apr. 17, 2014

(51) Int. Cl. G06F 19/00; F16M 11/04; F16M 13/00; A61B 17/06

(52) U.S. Cl. CPC G06F 19/3406; A61B 17/06114; F16M 11/04; F16M 13/00; F16M 2200/08

(57) ABSTRACT

The present invention is directed to a method, system, and apparatus for tracking the use, waste, and/or other characteristics of consumable medical items (e.g., surgical sutures) that are used during surgery. In one version of the invention, a sterile, disposable apparatus, denominated as a “landing pad,” is used to receive and position relative to one another: (1) an electronic device capable of taking digital images; and (2) the item being imaged (such as a sterile suture cartridge). The landing pad positions the electronic device and consumable medical item for digital imaging of the medical item. The resulting digital image is processed to identify a database record matching the imaged medical item. The identified record is then used to provide information about the imaged medical item (e.g., the count of individual surgical sutures contained in an imaged sterile suture cartridge). The information can be used for a variety of purposes, including, for example, an accurate accounting of all surgical sutures passed into the sterile field of an operating room for a scheduled surgery.

### BACKGROUND

Healthcare costs have been rising for years. Politicians, hospitals, health-care professionals, insurance companies, and others seek to lower these costs.

A business, seeking to reduce costs, often identifies and implements large-scale changes to its organizational design, products and services, office space, equipment, information services, and the like. Similarly, a hospital, seeking to reduce costs, might conceive and implement large-scale changes to its organizational design, services, equipment, information services, and the like. For example, many hospitals and healthcare professionals now offer certain surgical and diagnostic procedures on an outpatient basis, rather than an in-patient basis, saving time and money. Also, many healthcare organizations now employ computer hardware and software for client-relations management, rather than hard-copy files alone, to quickly and accurately enter, store, manage, and access patient information.

Many businesses, focusing on large-scale changes, forget the seemingly small ways by which an organization can reduce costs. In other words, many businesses forget to count paper clips. While savings from something as mundane as a single paper clip may seem vanishingly small, multiplying these savings across hundreds of thousands, or millions, of paper clips can add up to significant reductions in cost.

In some ways, surgical sutures (and other consumable medical items) may be thought of as one of a hospital's “paper clips.” A surgical suture is a medical device used to hold body tissue together after surgery. Typically a surgical suture includes a needle attached to a length of thread. A single surgical suture may cost from ten to one hundred U.S. dollars—a relatively small cost compared to the total cost of a single surgery. But every year, in the United States, doctors perform a hundred million or more surgical procedures, and most of these require surgical sutures, as well as other consumable medical items. Accordingly, any hospital or hospital system that more effectively uses surgical sutures could save hundreds of thousands, if not millions, of dollars. Unfortunately, hospitals lack the tools necessary to accurately track the number of surgical sutures that are used, or wasted, during a surgery (surgical sutures passed into the sterile field of an operating room during a surgery, but not used, must be thrown away—and are therefore wasted). And for want of these tracking tools, hospitals are not able to correlate suture use and waste with operating-room variables such as surgery type, surgeon identity, surgery start time, surgery duration, operating-room-personnel identities, hospital identity, etc.

Currently, hospitals have counting procedures to ensure that no surgical suture is unintentionally left inside a patient. Typically operating room personnel manually count both the number of surgical sutures that are passed into the sterile field of an operating room, and the number of surgical sutures remaining within the sterile field at the conclusion of the surgery (working to ensure that the number of sutures entering the sterile field equals the number of sutures within the sterile field at the conclusion of the surgery). Operating room personnel might use paper or a whiteboard to count the surgical sutures—a procedure that requires time, patience, and careful attention. As might be expected when people are working in high-stress situations, during surgeries lasting one to eight hours (and which might require tens to hundreds of surgical sutures of different types), mistakes are made. When a miscount is detected, which may occur once in every eight surgeries, the operating team must go through the extra time and expense of correcting the miscount. These extra steps might include: operating room personnel searching for the missing suture (e.g., using a metal detector); or x-raying the patient to ensure that a surgical suture was not unintentionally left inside the patient. Some health-care systems have chosen to x-ray every surgical patient to ensure that no surgical suture is unintentionally left within a patient.

In summary, the typical procedures used to count surgical sutures for health-and-safety purposes are subject to error, and implicate extra costs, both in time (e.g., the time required by operating room personnel to manually count and track surgical sutures; the time required for remedial steps if a miscount is detected), and money (e.g., x-rays to ensure that a suture needle is not unintentionally left in a patient). Furthermore, these conventional health-and-safety-counting procedures do not readily provide ways of correlating surgical suture use, and surgical suture waste, with other variables such as surgery type, surgeon identity, etc. Why this is so requires a discussion of the marketplace for surgical sutures, and the nature of the packaging of these sutures.

Surgical sutures come in a variety of shapes, sizes, and materials of construction. There are literally thousands of different kinds of surgical sutures. Also, hospitals and health-care systems purchase surgical sutures from hundreds of different suture manufacturers from around the world. Furthermore, suture manufacturers package surgical sutures in different ways. A typical package configuration for surgical sutures includes three levels of packaging: (1) a non-sterile carton or box, often made of cardboard, that contains a plurality of non-sterile suture packages; (2) a plurality of non-sterile suture packages (i.e., the external surface of the non-sterile suture package is not sterile), each non-sterile suture package containing a sterile suture cartridge (e.g., each non-sterile suture package may include a plastic tray within which the sterile suture cartridge is placed; typically a peelable foil, attached to the perimeter of the non-sterile suture package, overlays the sterile suture cartridge contained therein, and ensures that the sterile suture cartridge remains sterile until passed into the bounded sterile field within which a surgical procedure is performed); and (3) a sterile suture cartridge, which may contain one or more surgical sutures. While the non-sterile carton or box may include a bar code or other label that can be scanned for information (e.g., information identifying the surgical suture type and count), the non-sterile suture package and sterile suture cartridge contained therein usually have no such bar code or optically readable label.

Because there are thousands of different types of surgical sutures, available from hundreds of suture manufacturers worldwide, each with potentially different packaging designs and packaging counts (many of which lack an optically readable bar code or label), one or more of the surgical sutures, suture cartridges, and suture packages must be counted manually by hospital personnel, not only for health-and-safety purposes, as described above, but also for inventory purposes. For example, hospital personnel might use a hand-held bar-code reader to count the type and number of sutures contained in packages the include a bar code, and then rely on hand-written tallies of the surgical cartridges that are passed into the sterile field of an operating room. Just as manual counts lead to error when seeking to ensure that a suture does not unintentionally remain within a patient, these same manual counts lead to error when seeking to track inventories of individual sutures. Furthermore, these manual counts do not typically address the difference between used sutures (i.e., sutures actually used during surgery) and unused sutures (i.e., suture cartridges passed into the sterile surgery field, but which contain one or more surgical sutures that were not used and, now being non-sterile, must be disposed of). At least one proposal to improve counting involves the attachment of some form of scannable label to medical items that lack a bar code. Unfortunately, this proposal would require the extra step, and cost, of designing and attaching such a label to packaging.

What is needed is an apparatus, method, and system for automating the procedure by which: surgical cartridges, or other consumable medical items, as received from manufacturers, are counted when passed into a sterile surgical field (i.e., the “in count” of surgical sutures); the procedure by which used surgical sutures are counted (i.e., the “out count” of surgical sutures, which corresponds to those surgical sutures that a surgeon actually uses to bind tissue in a patient; when finished suturing a patient, the corresponding needle is typically separated from the thread and inserted into a Styrofoam board); and the procedure by which unused sutures and suture cartridges are counted (i.e., those suture cartridges that are passed into the sterile surgical field, but which contain one or more unused sutures; unused sutures are denominated as “waste”). Further, what is needed is a process for correlating “in counts,” “out counts,” “waste,” and/or ratios thereof with other operating-room variables, such as surgery duration, surgery type, surgeon identity, and the like. Still further, what is needed is the hardware architecture, tangible computer-readable storage media, and databases needed to carry out these methods.

### SUMMARY

We have conceived a new apparatus, method, and system for automatically counting sterile suture cartridges and, therefore, the number and type of individual surgical sutures that enter the sterile field of an operating room; the number and type of individual surgical sutures that are actually used during surgery; and the number and type of individual surgical sutures that enter the sterile field but are not used. Our invention accommodates the wide variety of packaging configurations offered by hundreds of worldwide manufacturers. Also, our invention is not dependent on surgical sutures or packaging having a specific design or appearance, and is not dependent on the presence of a UPC bar code (i.e., a Universal Product Code/bar code) or other such inventory label for conveying information about the product. Furthermore, the invention may be used for a variety of other packaged, consumable medical items used during surgery, such as packaged knife blades, devices, implants (e.g., allograft, homograft, and synthetic), molecular and cellular products, and the like.

In one version of our inventive method, a plurality of suture-cartridge database records is compiled. Typically, each suture-cartridge database record corresponds to a unique suture-cartridge type or model number from a specific manufacturer. Each suture-cartridge database record generally includes: a computer-readable description or file corresponding to a digital image of the suture cartridge; a count of the number of individual surgical sutures contained in the suture cartridge; the identity of the manufacturer; the surgical suture type; and other such information. By making a digital image of each sterile suture cartridge passed into the sterile operating field before and during surgery, and using image recognition to match these digital images to a specific suture-cartridge database record, the number (and type) of all incoming surgical sutures may be tracked (and, as needed, displayed, such as the total “in-count” of sutures during a given surgery).

The number and type of surgical sutures entering the sterile field of an operating room is tracked as follows. During surgery, operating-room personnel make a digital image of each sterile suture cartridge that is passed into the sterile field of the operating room. Once a digital image of a sterile suture cartridge is made, a computer-readable description of the digital image is compared to the plurality of computer-readable descriptions of digital images contained in the suture-cartridge database. A match is found between the computer-readable description that characterizes the sterile suture cartridge passed into the sterile field of the operating room, and a computer-readable description characterizing a digital image contained in a specific suture-cartridge database record. An image-recognition search algorithm is used to locate the suture-cartridge database record that matches the suture cartridge imaged in the sterile field of an operating room. Because each suture-cartridge database record also includes a field corresponding to the number of individual surgical sutures contained in the corresponding suture cartridge, the total number of surgical sutures passed into the sterile operating field may be tracked, incrementally and in real time.

In addition to tracking the number and type of each sterile suture cartridge entering the sterile field (and, therefore, the cumulative total of individual surgical sutures entering the sterile field), the invention may be used to track the number of sterile suture cartridges not actually used during surgery. Once a non-sterile suture package is opened, the sterile suture cartridge contained therein must be used. If not used, then the sterile suture cartridge must be disposed of. Accordingly, the inventive method may be used to track, as waste, those surgical cartridges entering the sterile field of the operating room, but not used. Advantageously, the inventive method provides medical professionals with a way of analyzing the number and type of surgical sutures not used during surgery. Furthermore, the inventive method provides ways by which the use, and waste, of a particular type of surgical suture may be correlated with other surgery or operating-room variables such as surgeon name, surgery type, surgery duration, surgery start time, and other such variables.

Also, the inventive method and apparatus may be used to track and display the cumulative total of those surgical sutures that are actually used during surgery. Once a surgical suture is used to bind tissue, the needle and excess thread are severed from the bound tissue. The used needle is typically attached to a material or substrate (e.g., by inserting the needle into a Styrofoam block). In one version of the inventive method, a digital picture is taken of a Styrofoam block to which used needles are attached. The digital picture is then subjected to image analysis to determine the number of needles attached to the Styrofoam block.

In summary, the invention provides an automated way by which operating personnel may, during surgery, know: the number of surgical sutures entering the sterile field of an operating room; the number of surgical sutures actually used during the surgical procedure; and the number of surgical sutures not used during the surgical procedure. A simple algebraic equation may be displayed so that all operating personnel are apprised of the then-current tallies (e.g., in count=out count+waste; or, alternatively, only the in count and the out count are displayed, with the final tally of waste ensuring that all surgical sutures entering the field are accounted for). Furthermore, the invention provides ways by which suture use, and waste, may, as described above, be correlated with surgery variables of interest (e.g., surgeon name, surgery type, and the like). Also, prior to a given surgery, operating personnel may obtain information of interest. For example, operating personnel might determine the surgical suture type, or types, typically used by doctors for a given type of surgery. Or personnel assisting a surgeon may determine the surgeon's preferences for suture types, and counts, for a particular surgery.

An inventive landing pad is used to make digital images of sterile suture cartridges within the sterile field of an operating room. The inventive landing pad typically includes a base; a holder, attached to or part of the base, for receiving and positioning each sterile suture cartridge to be imaged; a bracket, attached to the base, for receiving and positioning an electronic device comprising a camera; and a support that connects the base to the bracket. In one version of the invention, the landing pad is configured to receive and position an electronic pad or tablet comprising both a touch screen and a camera. The bracket receives and positions the electronic pad so that the touch screen is available for manipulation and use during imaging of individual suture cartridges. In some versions of the invention, the bracket is adjustable so that the electronic pad may be positioned in one orientation when digital images of each suture cartridge are made (e.g., in a substantially horizontal orientation, with the camera eye facing downward); and in a second, different orientation for more convenient viewing by operating room personnel (e.g., in a substantially vertical, or inclined, orientation so that operating personnel may conveniently view, on the touch screen/display, the then-current in count, out count, and/or waste tallies of surgical sutures within the sterile field of the operating room).

In some versions of the invention, the landing pad, like surgical sutures and other packaged, consumable medical items, is disposable. In other versions of the invention, the landing pad is durable and autoclavable.

These and other representative embodiments of methods for counting sterile suture cartridges; methods for correlating use, or waste, of surgical sutures with other surgery variables, such as surgeon name, surgery type, surgery duration, and the like; and a landing pad for imaging sterile suture cartridges within the sterile field of an operating room, are described below. Likewise various representative versions of systems, processes, hardware architectures, and tangible computer-readable media for carrying out the invention are described.

### DRAWINGS

FIG. 1 shows one version of a conventional, multi-tiered container for a surgical suture.

FIG. 2 shows one representative version of an apparatus, system, and network of the present invention.

FIG. 3 shows one version of a processing system for use with the present invention.

FIG. 4 illustrates part of a representative process of the present invention (the remaining part of this same process is illustrated in FIG. 5).

FIG. 5 illustrates part of a representative process of the present invention (the remaining part of this same process is illustrated in FIG. 4).

FIG. 6 shows one version of a display and electronic device/processing system when configured for use in a system and method of the present invention.

FIG. 7 shows a side view of one version of a landing pad for use with the present invention.

FIG. 8 shows a top view of one version of a landing pad's lower bracket portion.

FIG. 9 shows a top view of one version of a landing pad's upper bracket portion (for those embodiments that include an upper bracket portion).

FIG. 10 shows a front view of one version of a landing pad for use with the present invention.

FIG. 11 shows a side view of one version of a landing pad for use with the present invention.

FIG. 12 shows one version of a suture-cartridge database record.

FIG. 13 illustrates one representative process in which counting options are received and executed.

FIG. 14 illustrates one representative process in which sterile suture cartridges entering the sterile field of an operating room are counted.

FIG. 15 illustrates one representative process in which sterile suture cartridges entering the sterile field of an operating room, but not used by the end of the surgery, are counted.

FIG. 16 illustrates one representative process in which used surgical sutures are counted.

FIG. 17 shows one version of a display and electronic device/processing system when configured for use in a system and method of the present invention.

FIG. 18 shows one version of a display and electronic device/processing system when configured for use in a system and method of the present invention.

### DESCRIPTION

The following paragraphs describe different, representative versions of the invention. Before describing the invention, however, certain background topics are addressed. These background topics include: the nature and importance of maintaining a sterile field in an operating room; and the nature of packaging of surgical sutures used in such sterile fields. After providing this background, an overview of a representative method and system for tracking the use and waste of surgical sutures is given, along with exemplary embodiments of an apparatus to be used when taking digital pictures of suture cartridges in a sterile field. Next, additional detail is provided describing both inventive methods for tracking surgical sutures, and the inventive apparatus for use in imaging suture cartridges (and used surgical sutures). Note, too, that the computer-readable processes and data structures used to carry out the inventive methods, embodied in various media such as random access memory (RAM), read-only memory (ROM), optical discs, magnetic storage media, and other such optical and magnetic computer storage technology, is described. Finally, details are given regarding inventive methods for correlating the use and waste of different types of surgical sutures with operating-room variables such as surgery type, surgeon identity, etc. Also, applicability of the inventive method, apparatus, and system to other packaged, consumable items passed into the sterile field of an operating room is discussed.

### BACKGROUND

### Sterile Field in an Operating Room

First, some background. Hospitals must work hard to prevent, or at least drastically reduce the chance of, various infections caused by microorganisms. This is especially true in an operating room during surgery, where a surgical patient's internal tissue or organs are exposed to contact with various instruments, equipment, surgical supplies, operating room personnel, and the like. Accordingly, hospitals typically establish an imaginary boundary around a surgical patient. This boundary encloses what is called a “sterile field” around the patient. Everything and everyone going across this boundary (i.e., into the sterile field), and near the surgical patient, is scrubbed and sterilized. Thus, for example, operating room personnel scrub their hands and forearms using anti-septic, germicidal, or other substances to destroy microorganisms present on their skin. These same personnel then don sterile gowns, gloves, and other clothing before entering the sterile field. Similarly, surgical instruments such as scalpels, clamps, and the like are sterilized (e.g., by autoclaving) prior to transfer into, and deployment in, a sterile field. Finally, as is discussed in the following paragraphs with respect to the packaging of surgical sutures, some surgical items or substances are provided in containers that maintain the sterility of the item or substance until the container is opened.

Because of the importance of maintaining the sterility of the sterile field, some equipment is typically kept outside the field, though used during surgery. So, for example, various kinds of electronic monitors cannot be scrubbed with germicidal substances, nor exposed to extreme heat (as occurs when an item is autoclaved). Accordingly, much of this equipment is not kept within a sterile field during surgery, but may be viewed by operating-room personnel working within the sterile field.

In summary, any innovator seeking to use electronic equipment to automatically track surgical items passing into a sterile field must anticipate, and address, the aforementioned constraints relating to maintaining the sterility of the field.

### Packaging of Surgical Sutures

Because a hospital must maintain a sterile field around a surgical patient during surgery, manufacturers providing items or materials for surgery must be cognizant of, and address, this sterility requirement. This is true for manufacturers of surgical sutures. As noted above in the Background section, there are literally hundreds of worldwide manufacturers of surgical sutures. And there are thousands of different types of surgical sutures (i.e., surgical sutures have different shapes, different cross sections, different sizes, different materials of construction [of both the needle and the thread attached to the needle], etc.). Also, many surgical sutures are offered in a tiered packaging system, like that described in the Background section. A typical package configuration for surgical sutures includes three levels of packaging: (1) a non-sterile carton or box, often made of cardboard, that contains a plurality of non-sterile suture packages; (2) a plurality of non-sterile suture packages, each non-sterile suture package containing a sterile suture cartridge (e.g., each non-sterile suture package may include a plastic tray within which the sterile suture cartridge is contained; typically a peelable foil, attached to the perimeter of the non-sterile suture package, overlays the sterile suture cartridge contained therein, and ensures that the sterile suture cartridge remains sterile until passed into the bounded sterile field within which a surgical procedure is performed); and (3) a sterile suture cartridge, which may contain one or more surgical sutures. While the non-sterile carton or box may include a bar code or other label that can be scanned for information (e.g., information identifying the surgical suture type and count), the non-sterile suture package and sterile suture cartridge contained therein often have no such bar code or optically readable label.

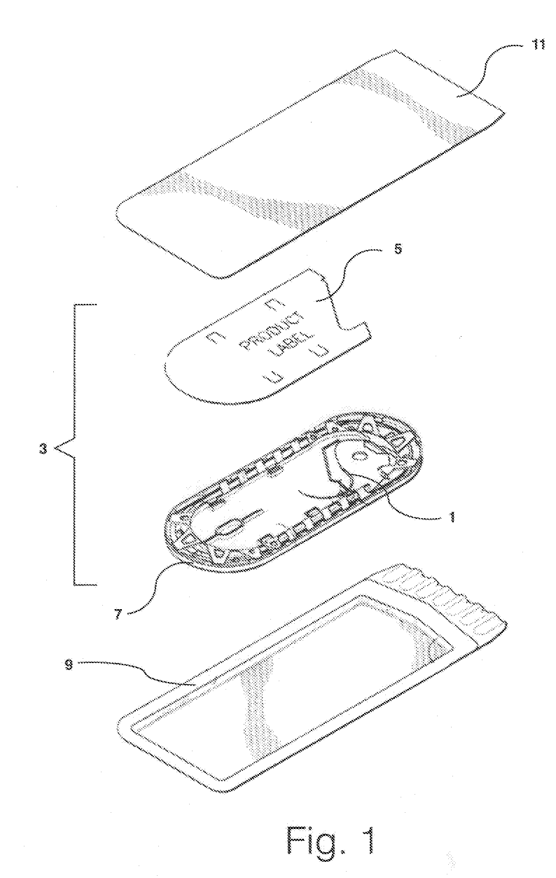

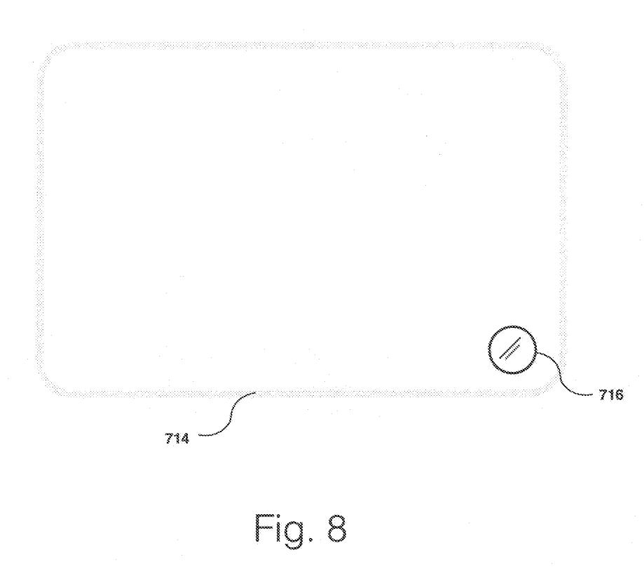

FIG. 1 (adapted from FIG. 8 in U.S. Pat. No. 5,833,055; U.S. Pat. No. 623,874; to Cerwin J. Robert, et al.; filed on 29 Mar. 1996, and entitled “Surgical Suture Package with Peelable Foil Heat Seal; this patent is hereby incorporated by reference in its entirety in a manner consistent herewith) shows one representative version of a combination of a non-sterile suture package and a sterile suture cartridge (with the sterile suture cartridge contained in the non-sterile suture package). A sterile surgical suture 1, which includes a length of thread attached to a needle, is contained within a sterile suture cartridge 3. The sterile suture cartridge typically includes a product label 5, often made of paperboard, attached to a suture support 7. Typically the product label includes some indicia of the surgical suture contained therein, and may include: the name of the manufacturer or supplier; the name and/or model number of the surgical suture; a depiction of a cross-sectional view of the needle; a depiction of a side view of the needle; numerical specifications of size and length (e.g., length of thread attached to needle; metric and/or non-metric gauge sizes of needle); textual descriptions of the surgical suture; method (or methods) of sterilization; etc. Usually the product label on the sterile suture cartridge does not include a Universal Product Code—i.e., the widely adopted barcode symbology used to track items (for example, items sold in retail stores).

The sterile surgical suture, attached to and supported by the sterile suture cartridge, is itself contained within a non-sterile suture package. For the embodiment depicted in FIG. 1, the non-sterile suture package includes a tray 9, which is configured to receive the sterile suture cartridge; and a peelable foil 11, which is releasably attached to the tray 9. The sterility of the suture cartridge is maintained by this packaging configuration until the peelable foil 11 is removed. Typically, operating room personnel outside the sterile field remove the peelable foil from the non-sterile suture package and carefully, without touching the sterile suture cartridge, “flip” the sterile suture cartridge from outside the sterile field onto a sterile tray within the boundary of the sterile field. Once opened in this way, the sterile surgical suture, attached to the now-exposed sterile suture cartridge, must be used during the surgery . . . or thrown away. The exposed suture cartridge cannot be stored for future use, or re-used.

As noted above, hospitals typically use paper and pencil, whiteboards, or other equipment to track surgical sutures. The hospital's primary goal is to make sure that a surgical suture is not left inside a patient. If there is an error-if operating room personnel cannot account for each and every surgical suture that is passed into the sterile field-then personnel must search for the missing suture (or sutures). At the extreme, personnel will x-ray the patient to ensure that a surgical suture was not mistakenly left inside a patient.

### Overview of Representative Versions of Inventive Method, Apparatus, and System

### Overview of Landing Pad

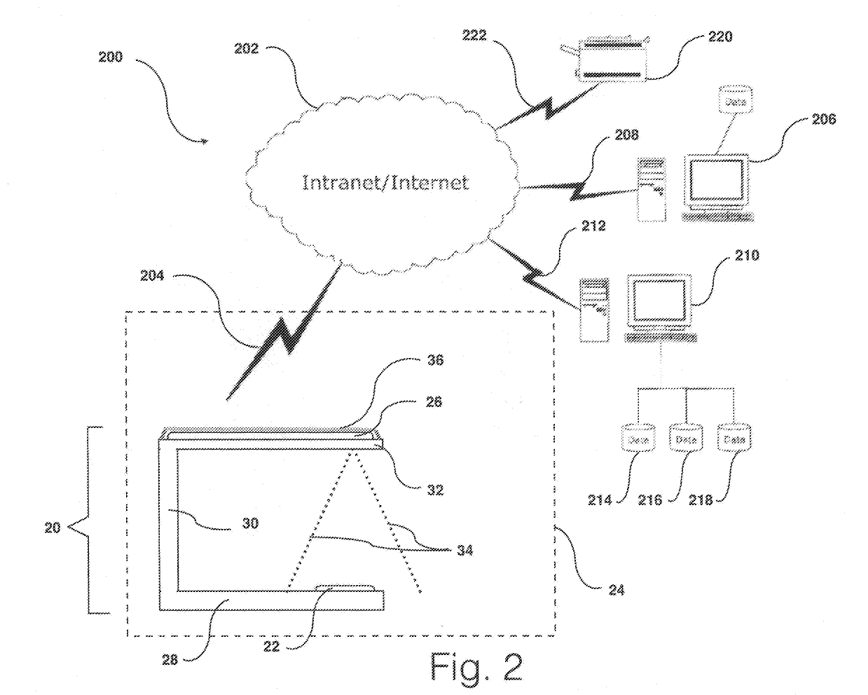

FIG. 2 shows one representative version of an apparatus, and system, of the present invention (FIG. 2 includes a side view of the apparatus): a disposable or durable landing pad 20 is used to position a sterile suture cartridge 22 within the sterile field 24 of an operating room for digital imaging by an electronic device/processing system 26 that includes a digital camera, such as an electronic tablet, smart phone, or other such device (note: the rectangular, dashed border signified by 24 represents the imaginary boundary defining a sterile field around a patient in an operating room—FIG. 2 does not show all of the operating room personnel and other equipment inside the boundary of the sterile field—instead, for purposes of FIG. 2, boundary 24 shows that the landing pad 20 is used inside a sterile field when imaging sterile suture cartridges).

While the exact configuration of the landing pad, as described below, varies, it typically includes a base 28, a support 30, and a bracket 32. For the representative version of the landing pad shown in FIG. 2, a sterile suture cartridge 22 is placed on the base 28. Typically the base includes clips, clamps, a printed grid, a recess or depression within the base, or other structure to aid a user in positioning the suture cartridge below the camera eye of the electronic device used to digitally image the suture cartridge. These features are not shown in FIG. 2.

The support 30 connects the base 28 to the bracket 32. As is discussed below, the support may be a single piece, or multiple pieces (e.g., two, three, four or more pillars supporting the bracket 32 above the base 28). Furthermore, the support may be a separate component that is attached to the base and/or bracket, or may be integrally formed with one or both of the base or bracket. A person of skill will recognize that various designs may be used, so long as the landing pad positions a camera—typically part of an electronic device—in a spaced-apart relationship relative to a suture cartridge so that digital pictures of the suture cartridge can be taken.

The bracket 32 is designed to receive and position an electronic device having a digital camera, such as an electronic tablet, relative to a suture cartridge so that the device may be used to produce a digital picture of the suture cartridge. The bracket, then, must provide a clear line of sight between the eye of the digital camera and the suture cartridge being imaged. As discussed below, the bracket may have an opening, or a transparent portion, below the eye of the digital camera associated with the electronic device. In FIG. 2, dotted lines 34 represent a clear field of view below the eye of the digital camera such that digital images of the suture cartridge may be taken by the electronic device (note: the lines are not meant to precisely define the field of view; only to show that bracket 32 must allow the electronic device 26 to take pictures of the suture cartridge 22). The bracket may assume different configurations. For example, the bracket may be a clam-shell-like design that can receive and enclose an electronic device such as an electronic tablet (this exemplary configuration is shown and described below). Or, for the representative embodiment shown in FIG. 2, the bracket may consist of an open tray that can receive and position the electronic device. A transparent, sterile plastic layer 36 is placed over the electronic device 26 to ensure that the integrity of the sterile field is maintained. This plastic layer may adhere to at least some portion of the electronic tablet; to at least some portion of the bracket (as is depicted in the representative version of the invention shown in FIG. 2); or both. The layer is transparent so that a user of the landing pad can view the screen of the corresponding electronic device. The layer is sterile so that the non-sterile electronic device, once positioned on the landing pad's bracket, may be isolated from contact with operating personnel and equipment within the sterile field (i.e., the sterile, transparent plastic, once placed on the electronic device and/or bracket, is interposed between personnel using the electronic device, and the device itself). Finally, the plastic layer is typically flexible and sufficiently thin so that operating room personnel within the sterile field of the operating room may make use of any touch-screen capabilities of the electronic device.

### Overview of Representative Network and Representative Processing Systems

FIG. 2, in addition to depicting the inventive landing pad, also shows a network 200 typically used with the inventive system for tracking suture cartridges during surgery; for generating and transmitting suture-use analytics for use in promoting hospital efficiency; for maintaining the inventory of suture cartridges; etc. The network 200 typically includes an Intranet and/or Internet 202 that connects various processing systems. The network allows for the exchange of data between the processing systems. One skilled in the art will recognize that processing systems are personal computers, laptop computers, electronic tablets, smart phones; system computers, routers, and other such devices used to process digital data. One skilled in the art will also recognize that the exchanged data may represent computer-readable software instructions; computer-readable files representing text, numbers, images, audio, and the like; etc.

In the representative network 200 shown in FIG. 2, an electronic tablet 26, positioned on the landing pad 20 within the sterile field 24, may be connected to the Intranet and/or Internet via path 204. A person of skill in the art will recognize that path 204 (and other paths connecting any other processing unit to network 200) may be via a wireless connection (e.g., using Wi-Fi brand networks), or a wired-in connection (e.g., an Ethernet, or other, cable). Given that an electronic tablet is a processing unit, in some versions of the invention the tablet itself may be used to carry out representative inventive suture-cartridge tracking methods, and generate and display suture-use analytics, without connecting to a network such as network 200. Typically, however, at least some aspects of the invention will be carried out using a network such as the representative version shown in FIG. 2.

Often representative networks used with the inventive system will include a data-transfer server 206, connected via path 208 to the Intranet and/or Internet 202, that controls data transfers between processing systems connected to the Intranet and/or Internet. A router is one example of such a data-transfer server.

The representative version of a network shown in FIG. 2 also includes a database server 210 connected via path 212 to the Intranet and/or Internet 202. The database server is a processing system that maintains various databases that may be accessed using the invention. For example, the database server (or servers) may maintain one or more of a suture-cartridge database 214, a suture-use analytics database 216, and other databases that may be used in conjunction with the invention (e.g., an inventory database 218). One of skill will understand that two or more databases need not reside on the same processing unit (and, in fact, one or more databases may reside on the processing unit used to image the suture cartridge). Also, one of skill will understand that a user of the inventive system may compile information to populate each of the individual databases. Alternatively, a third-party provider may compile and/or populate the individual databases.

As described in more detail below, the suture-cartridge database 214 stores records containing information relating to the different suture-cartridges that may be passed into the sterile field of an operating room during surgery. Individual suture cartridges passed into the sterile field of an operating room are imaged as described generally in the present application. Image-recognition analysis is used to search and retrieve a suture-cartridge database record, or information stored in such record, that matches the imaged suture cartridge. The matched record includes the count of the number of individual sutures contained in the suture cartridge. This count is then used when automatically tallying the number and type of sutures passed into the sterile field; and the number and type of any sutures passed in to the sterile field but not used.

As detailed below, the suture-use analytics database 216 stores records containing information relating to suture use for completed surgeries. This database may include: the number and type of each suture passed into the sterile field during a surgery, and used; the number and type of each suture passed into the sterile field during a surgery, and not used (and therefore, wasted); for each suture type, the percentage of sutures wasted; for each suture type, the cost of the wasted sutures; etc. In addition to information characterizing suture use, the suture-use analytics database also includes information on operating-room or surgery variables such as the name of the surgeon (or surgeons); the names of other operating personnel; the operating room number or other designator; the start time of the surgery; the duration of the surgery; the number of shifts of operating room personnel working during the surgery; the name of the hospital; etc. Information can be retrieved from these various records to generate reports relating suture use to other surgery variables.

Other databases that may be used in conjunction with the invention include an inventory database 218. One of ordinary skill will recognize that each of these databases may be well-known database applications that are populated, and, as needed, modified for use with the present invention. Or, alternatively, the database may be custom made for use with the present invention.

The representative network 200 shown in FIG. 2 may also include a printer 220 or other output device connected to the Intranet and/or Internet 202 via path 222.

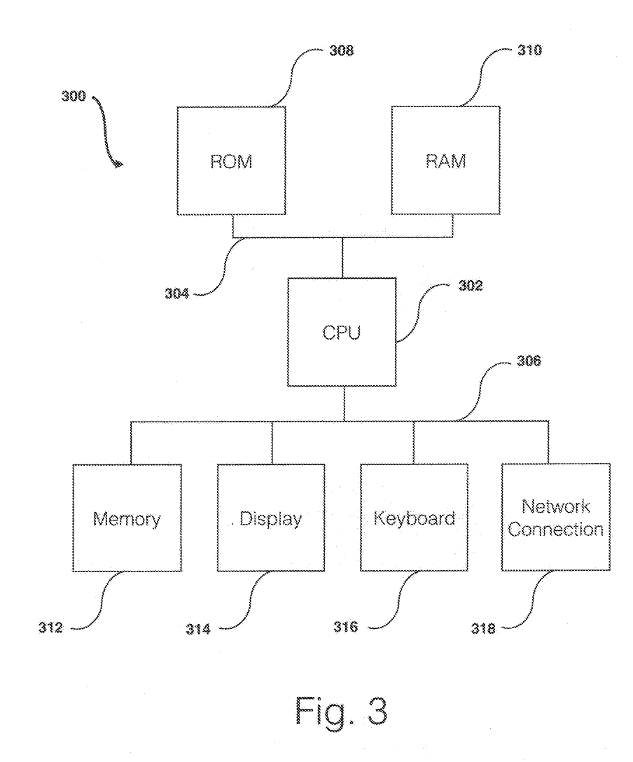

FIG. 3 illustrates generally a representative embodiment of a processing system. One skilled in the art will recognize that most if not all of the devices connected to network 200 in FIG. 2 includes a processing system. The nature of each device, and its configuration, may vary. One representative version of a processing system, processing system 300, has a central processing unit (“CPU”) 302. Central processing unit 302 is a processor, microprocessor, or any combination of processors and microprocessor that execute instructions (with these instructions typically stored in tangible, computer-readable storage media, or memory) to perform an application. Central processing unit 302 is typically connected to a memory bus 304 and an input/output (“I/O”) bus 306. A person of skill will recognize that some CPUs include high-speed memory-typically denominated as a cache-built directly into the CPU.

A non-volatile memory, such as read-only memory (“ROM”) 308 is connected to CPU 302 via the memory bus 304. Read-only memory 308 typically stores instructions for initialization and other system commands of processing system 300, and various firmware applications (i.e., instructions for hardware components such as hard disks, DVD drives, etc.). One skilled in the art will recognize that any memory that cannot be written to by CPU 302 may be used for the functions of ROM 308.

A volatile memory such as random-access memory (“RAM”) 310 is also connected to CPU 302 via memory bus 304. Random-access memory 310 stores instructions for all processes being executed (as well as data operated upon by the executed processes). One skilled in the art will recognize that various types of memories including, but not limited to, dynamic random-access memory, static random-access memory, and phase-change memory may be used as a volatile memory; and that memory caches and other memory devices (not shown) may be connected to memory bus 304 and/or CPU 302.

Peripheral devices or other processing-system components including, but not limited to, memory 312, display 314, I/O device 316, and network connection device 318 are connected to CPU 302 via I/O bus 306. Input-output bus 306 carries data between the device and CPU 302. Memory 312 is a device for storing data on media. Some examples of memory 312 include read/write compact discs (CDs), and magnetic disk drives. Display 314 is a monitor or display and associated drivers that convert data to a display. Input-output device 316 is a keyboard, touch screen, pointing, voice-recognition, or other device that may be used by a user to input data (note: a touch screen is both an input-output device and a display). Network device 318 is a modem, Ethernet, or other component that connects processing system 300 to a network.

One skilled in the art will recognize that the exact configuration and devices connected to each processing system in network 200 may vary depending upon the operations that the processing system performs in the network. Persons of skill will recognize that a processing system will typically include a control unit, such as a CPU, for orchestrating what is done by a processing system. The control unit reads and executes program instructions stored on data memory and/or inputted from an external input device (e.g., a tangible computer-readable storage medium that includes instructions stored thereon which, when executed by a computer device/electronic device/processing system, causes the processing system to perform operations or process steps, like those described in the present application). Often the processing system includes an arithmetic and logic component for any math or logic to be performed as part of the execution process. Also, processing systems include input (e.g., keyboard, mouse, trackball, voice recognition, touch screen, etc.) and output (e.g., display, printer, audible voice or signal, etc.) components.

A person of skill will appreciate that processes of the present invention may be implemented as computer programs stored in various memory devices and/or received through a communications interface or network connection. These computer programs, when executed, enable one or more processing systems to perform features and processes of the present invention. A person of skill will recognize that when the invention is implemented using software, the software may be stored on a computer program product and loaded onto a processing system using a removable storage drive, hard disk drive, or network connection. For example, a computer program product embodying features of the invention may be downloaded to a processing system over a communications path (e.g., the downloading of a computer program product using the iTunes®-brand application program, or from some other entity using other programs or software).

### Overview of Representative Process

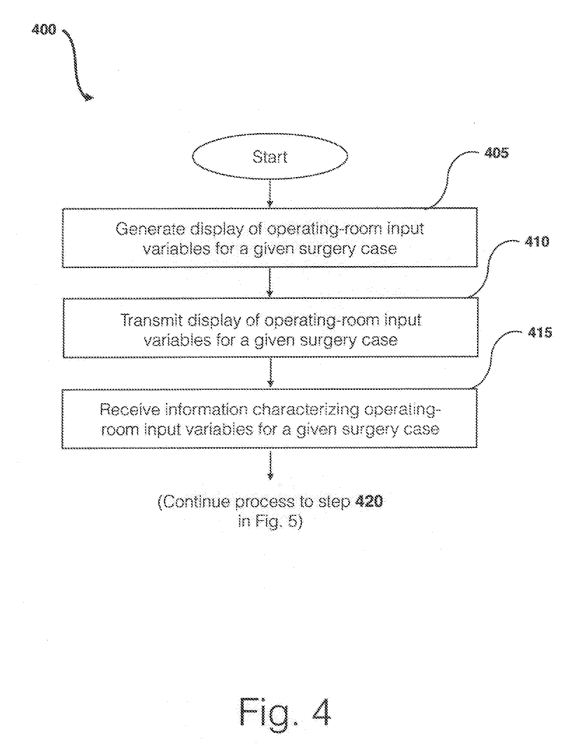

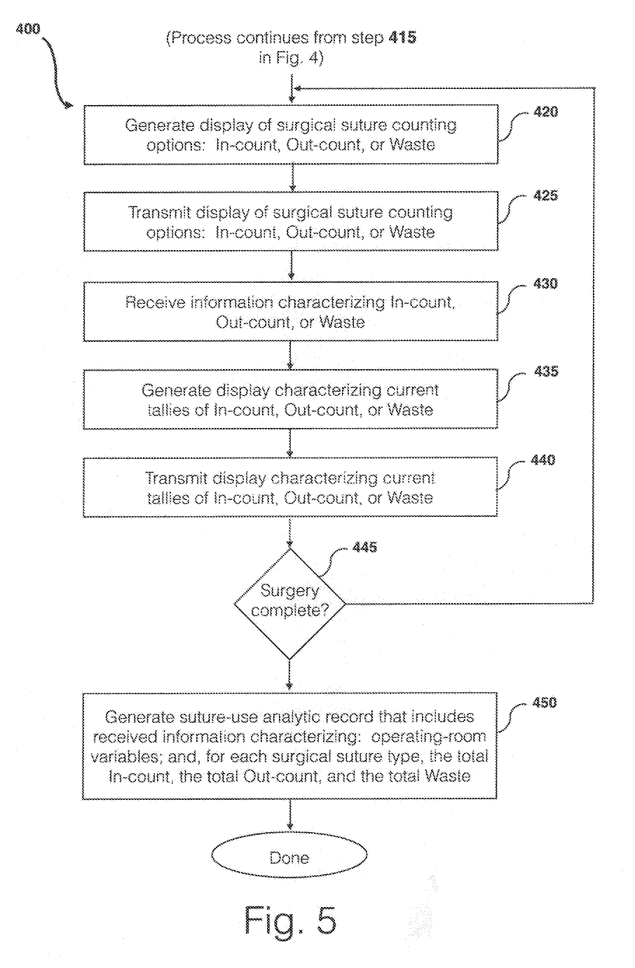

FIGS. 4 and 5 illustrate a flow diagram of a representative process 400 of the present invention. Generally, the process 400 is executed to provide an interface by which processing system(s) are used to track the number of sutures entering, and used in (or not used in) a sterile field for a given surgery; as well as the processing system(s) by which suture-use analytic information is compiled. Furthermore, process 400 encompasses other processes used to count: suture cartridges passing into the sterile field; suture cartridges passed into the sterile field but not used; and used sutures (depending on the counting option selected by a user). Additional detail on these processes is given below.

FIG. 4 illustrates the first steps of process 400. First, in step 405, a display is generated by the processing unit of the processing system executing process 400. The display includes at least some of the operating-room input variables for a given surgery case. These operating-room input variables, or surgery variables, may include: name of surgeon (or names of surgeons); names of other operating room personnel; surgery type; surgery date; expected surgery start time; expected surgery end time; expected surgery duration; operating room number or other designation; hospital name or designation; etc. Typically the display will include drop-down menus, open fields for data entry, and the like so that a user of the process can select, type, or otherwise input information for one or more of the surgery input variables.

In step 410, the display is transmitted. If a processing system is accessing process 400 via a network connection (and if the accessing processing system is different from the processing system executing at least some of the steps of process 400, such as steps 405 and 410), then the processing system executing at least some of the steps of process 400 transmits the display to the accessing electronic device/process system). If the processing system accessing and executing process 400 are one in the same, then the display is transmitted to the display device of processing system. One skilled in the art will recognize that different instructions may be needed to generate the display depending on which device receives the display.

In step 415, information characterizing at least some of the operating-room input variables is received for the given surgery case. It should be noted that the received information might be incomplete (i.e., a user entering information in response to the transmitted display may not be able to enter or select all of the numeric and alphabetic values for the operating-room input variables before the end of the surgery case). Also, while the representative process depicted in FIGS. 4 and 5 shows steps 405, 410, and 415 as occurring before subsequent steps in which suture cartridges and sutures are counted, the invention encompasses versions in which information characterizing operating-room input variables is not received until after a surgery is over.

In step 420 (see FIG. 5), a display is generated by the processing unit of the processing system executing process 400. In representative versions of the present invention, this display includes three counting options that may be selected by a user of the system: (1) an option typically denominated as the “In Count” option: an option in which a sterile suture cartridge, having been passed into the sterile field of the operating room, is imaged, with a matched and retrieved suture-cartridge database record (or information retrieved from a field contained in the database record) providing the count of the number of individual sutures contained in the imaged sterile suture cartridge (and, at the same time, providing other information about the imaged sterile suture cartridge, such as the manufacturer; suture model number or type; etc.); (2) an option typically denominated as the “Used” option: an option in which the total number of used sutures is determined by image analysis of an image of used needles attached to, for example, a Styrofoam block (an option in which a suture-cartridge database record is typically not retrieved; instead, image analysis is used only to determine the total number of used needles); and (3) an option typically denominated as the “Waste” option: an option in which a sterile suture cartridge, having been passed into the sterile field of the operating room, but not used, is imaged, with a matched and retrieved suture cartridge database record (or information retrieved from a field contained in the database record) providing the count of the number of individual sutures contained in the imaged sterile suture cartridge that was not used (and, at the same time, providing other information on the imaged sterile suture cartridge, such as the manufacturer; suture model number or type; etc.). More detailed processes characterizing these options are discussed in more detail below.

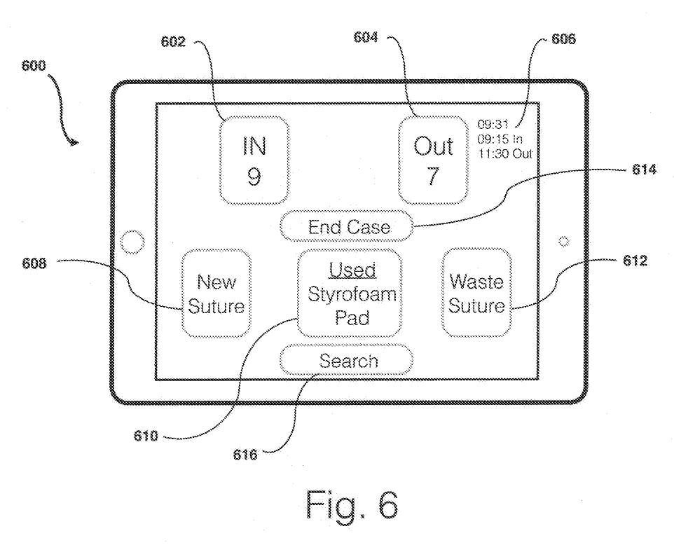

One example of a display of the available counting options, as well as then-current tallies of the In-count and Out-count of surgical sutures, is shown in FIG. 6. FIG. 6 shows the top view of an electronic tablet/processing system 600. As discussed above, the electronic tablet will typically be placed in the inventive landing pad, which serves to receive and position both the electronic device and a suture cartridge (or Styrofoam block containing used sutures) so that the electronic device can generate a digital image of the suture cartridge (or Styrofoam block containing used sutures). Furthermore, the landing pad serves to interpose sterile materials between the non-sterile electronic device and operating-room personnel in the sterile field of an operating room. For the representative display shown in FIG. 6, a process has transmitted a display to the electronic device/processing system so that the display includes: the in-count 602 of individual surgical sutures entering the sterile field of the operating room, denominated by the word “IN” over the then-current numeric count of sterile sutures entering the sterile field, which in the depicted example is “9”; and the out-count 604 of individual surgical sutures that have actually been used during surgery, denominated by the word “OUT” over the then-current numeric count of used surgical sutures, which in the depicted example is “7”. For the representative display shown in FIG. 6, timing information 606 is provided for the particular surgery underway. For the example depicted in FIG. 6, the time is “09:31”(note: hospitals often use military time to avoid confusion); the expected start time of the surgery was “09:15”; and the anticipated end time of the surgery is “11:30”. Persons of skill will recognize that many other combinations of calendar, time, and duration displays are possible. FIG. 6 also provides a representative example of the counting options that may be provided in a display, including: an in-count option 608, in this example denominated as “New Suture”; an out-count option 610, in this example denominated as “Used/Styrofoam Pad”; and a waste option 612, in this example denominated as “Waste Suture”. If the visual display is presented on a screen that also functions as a touch screen for selecting or inputting information, then the process may receive information through a user's selections or inputs via the touch screen. Accordingly, persons of skill will recognize that a process may receive information from a user touching or otherwise activating a given region on the touch-screen display (e.g., by a user touching the iconic button signifying the New Suture/in-count counting option 608). The representative display in FIG. 6 also includes an iconic button denominated as “End Case” 614, and an iconic button denominated as “Search” 616. A user touching or activating the end-case button 614 causes relevant processes to end the counting of surgical sutures for a particular case, and to compile information relating to suture use and other surgery variables. A user touching or activating the search button 616 causes relevant processes to search for information requested by the user.

Returning, then, to FIG. 5: in step 425, the generated display is transmitted to the same or different processing system, depending on whether the same or different processing system is accessing and executing some or all of process 400.

In step 430, and in response to a user selecting one of the available options (i.e., the In-count option, the Out-count option, or the Waste option), a suture cartridge (or used needles) are digitally imaged (e.g., using the landing pad apparatus discussed above), with the digital image serving to determine the selected count through image analysis (e.g., image-recognition analysis, optical-character recognition, or other such image analysis as is discussed in more detail below). Information characterizing one of these counting options is received in order to update the then-current tally of the corresponding option. More detailed processes characterizing this step are discussed in more detail below.

In step 435, a display is generated characterizing the current tallies of the In-count, the Out-count, Waste, or some combination thereof. And in step 440, the generated display is transmitted to the same or different processing system, depending on whether the same or different processing system is accessing and executing some or all of process 400. One example of a representative display of the then-current tallies of one or more of the In-Count, Out-count, and/or Waste is shown in FIG. 6.

In step 445, process 400 determines whether a quit, exit, surgery complete, end case, or similar command is received. If so, then process 400 proceeds to step 450. If not, then process 400 returns to generate and transmit a display regarding surgical suture counting options (or, alternatively, returns to the process immediately before step 430, ready to receive information characterizing a selected counting option). At a surgery case's end, process 400 executes step 450, in which a suture-use analytic record is generated for the surgery. This record includes received information characterizing surgery variables (e.g., surgeon's name, etc.) and information received characterizing the use and waste of surgical sutures entering the sterile field. Aggregated suture-use analytic records provide information needed to evaluate trends and correlations relating to suture use and waste.

### Additional Detail on Representative Versions of the Landing Pad

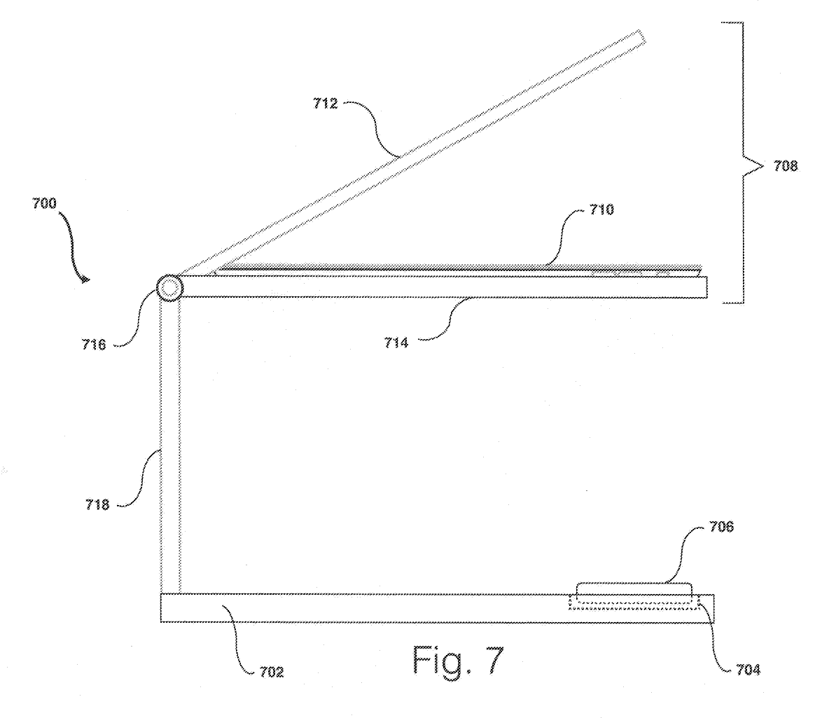

As discussed elsewhere, the inventive method, apparatus, and system are used to automatically track the count of surgical sutures. One aspect of the invention is an apparatus used to facilitate production of a digital image of each sterile suture cartridge passed into the sterile field of an operating room. A side view of a representative version of such an apparatus, denominated a “landing pad” 700, is shown in FIG. 7 (note: the designator 700 and corresponding arrow signify the landing pad only, not the electronic device 710 and suture cartridge 706, which are also shown, and discussed below, when explaining the features of the landing pad). This particular version of the landing pad includes a base 702; a holder 704 attached to or integrally formed in the base, the holder configured to receive and position a sterile suture cartridge 706; and a bracket 708 attached to the base, the bracket being configured to receive and enclose, within the sterile field, an electronic device 710 having both a camera, and an electronic screen that serves both as a visual display and as a touchscreen (e.g., for inputting information; manipulating and controlling the display; etc.). (Note: for the representative version of the landing pad shown in FIG. 7, the designator 704 signifies a dotted line defining a recess or depression integrally formed in the base, the recess or depression being of a size and shape configured to receive the range of suture cartridge sizes to be used during surgery; also, the designator 706 signifies a suture cartridge, a portion of which is drawn with a solid line to represent that portion of the suture cartridge visible from the side view depicted in FIG. 7—and a portion of which is drawn with a dotted line to represent that portion of the suture cartridge resting in the recess or depression and below the upper surface of the base, and therefore not visible from the side view depicted in FIG. 7; finally, designator 708 and the corresponding brace signify a bracket that includes an upper portion 712 and a lower portion 714, attached by a hinge 716, that may be opened and closed in a clam-shell-like fashion to receive and enclose an electronic device—in FIG. 7 the bracket is shown in a partially open position). In this particular version of the landing pad, a support 718 joins the bracket 708 (i.e., the lower portion of the bracket; the upper portion of the bracket; or both) to the base 702.

The landing pad depicted in FIG. 7 can be durable, or disposable. If disposable, then the landing pad is made and packaged so that the landing pad remains sterile until use. After operating room personnel use the landing pad for a particular surgery, the pad is disposed of. To minimize shipping and storage costs, the landing pad is typically designed so that it may be shipped in a collapsed form, and then assembled or deployed for use in the sterile field of an operating room. For example, in one version of the invention, the landing pad is made of separate components that may be assembled before use in an operating room.

In another version of the invention, the disposable landing pad is made of one or more components joined together by hinges or other mechanical attachments that allow the components to be positioned at different orientations relative to one another (e.g., if a hinge is used, a manufacturer of the landing pad selects one position for shipping by rotating joined components about the hinge so that the components are substantially parallel to one another; and operating-room personnel select another, different position before surgery, by rotating these same components about the hinge so that the components are substantially perpendicular to one another). For versions of the landing pad that are disposable, any conventional method and design by which the components of the landing pad may be shipped and stored in a sterile, collapsed or unassembled form; and then assembled, or re-positioned, before use in the sterile field of an operating room, may be used.

Typically the disposable landing pad is composed of plastic or other polymeric materials. The landing pad, or components thereof, may be made using a variety of injection-molding or other manufacturing techniques. As noted above, versions of the landing pad that are disposable are sterilized prior to packaging. Any conventional method for sterilizing parts or products during manufacturing and/or packaging processes may be used to sterilize the disposable landing pad, or components thereof, prior to its packaging and shipment (e.g., radiation, ethylene oxide, etc.).

FIG. 7 shows a substantially rectangular base 702 and bracket 708, joined by a support 718. Each of these three components may be of other shapes. For example, the base 702 may be a square, oval, circle, or some other shape, so long as the resulting landing pad is stable during use (i.e., with an electronic device positioned in or on bracket 708). Similarly, the support 718 may be a square or other shape, so long as the deployed landing pad, as noted above, is stable during use. The support may be a single piece, or it may be composed of multiple pieces (e.g., individual pillars or columns that attach to the base at, for example, its corners; and which likewise attach to the bracket at its corners; other variations are possible, so long as the resulting combination of a base, a bracket, and a support results in a stable landing pad during use). The same may be said of the bracket 708, along with the additional proviso that the bracket allow for the electronic device's camera to function, with a clear line of sight from the eye of the camera to any surgical suture cartridge that is being imaged. It should be noted that the phrase “clear line of sight” encompasses placement of a transparent material between the camera eye and the suture cartridge. Furthermore, the bracket must include, or allow for the placement of, a transparent portion that allows the electronic device's screen to be seen, and manipulated (via the touchscreen features of the electronic screen)—while preserving the integrity of the sterile field. It should be noted too, that reinforcing members may be used in some versions of the landing pad to enhance stability (e.g., a pair of diagonal reinforcing members—one on each side of the landing pad—in which one end of each diagonal member is attached at, for example, one side of the support member; and the opposing end of each diagonal member is attached at, for example, one side of the bracket's lower portion).

The design of the landing pad reflects the size and weight of various electronic devices that may be used in conjunction with the landing pad. For example, mobile phones (smart phones), electronic tablets, and other such devices may be used with the landing pad. The screen sizes of these various devices typically range from about 3 inches to about 13 inches or so (measured diagonally), with the electronic device sized to accommodate the screen. The weight of these same devices may range from less than 0.5 pounds to 2 pounds or more. Accordingly, a line of disposable landing pads of different dimensions may be designed, and offered to accommodate, both the size and weight of the various electronic devices that are available. Alternatively, the base and support may be of the same size, with different bracket designs and sizes available to accommodate the array of electronic devices that are available. Also, the interior of the bracket may be terraced—i.e., have a sequence of differently-sized depressions, with each depression accommodating a differently sized electronic tablet.

The bracket 708 shown in FIG. 7 is configured to receive, and enclose, an electronic device so that: (i) the device's camera may be used to take digital pictures of sterile suture cartridges; and (ii) the device's electronic screen may be used to display information (e.g., the current tally of surgical sutures passing into the sterile field), and to manipulate and retrieve information (e.g., to take digital pictures of sterile suture cartridges passed into the sterile field; to retrieve and display a particular surgeon's preferences for the number and specific types of surgical sutures to be made available for a particular type of surgery; etc.). As noted above, the representative version of a bracket shown in FIG. 7 may be thought of as a clamshell, with a lower bracket portion 714 attached to an upper bracket portion 712 by a hinge 716.

FIG. 8 shows a top view of the lower bracket portion 714 with a transparent window 716 (note: for simplicity, neither the upper portion of the bracket, nor any hinge or other component, is shown in FIG. 8). An electronic device placed in the bracket is positioned so that the device's camera eye is aligned with transparent window 716. The transparent window may be of any size, and at any location, on the bracket, so long as the electronic device contained therein is able to take digital pictures of a suture cartridge. In some versions of the invention, a more substantial portion of the bracket is made of a transparent polymeric material such that there is no need for a localized transparent window 716. Alternatively, opening 716 may be just that: an opening in the lower bracket portion, without any transparent or other plastic interposed between the electronic device's camera eye and the surgery item being imaged. Also, as noted above, in some versions of the invention, the landing pad design is adapted to be used with a variety of electronic devices. Because design features of these devices differ (e.g., by size, weight, camera eye placement, etc.), the bracket design, including the placement and size of any transparent window, may also differ. A person of skill will recognize that a variety of bracket configurations may be deployed to accommodate the variety of electronic devices/processing systems available for use with the invention.

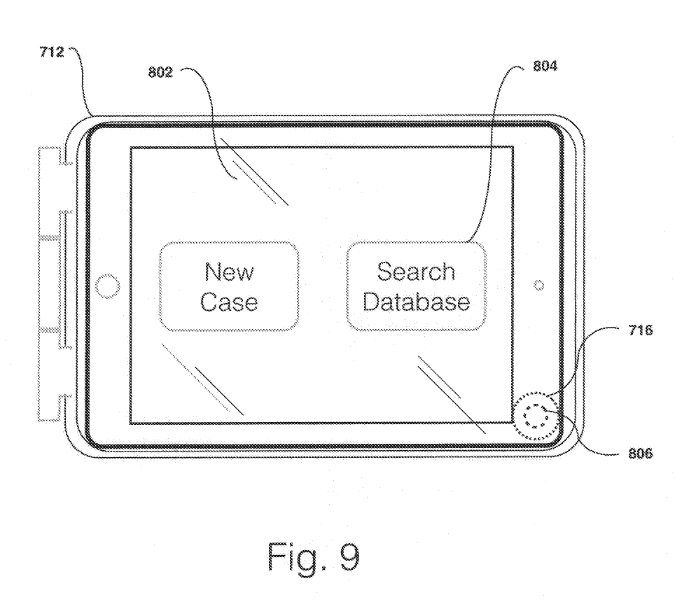

As noted above, the bracket design also allows operating room personnel to view information displayed on the electronic screen of the device contained therein; and to manipulate the touch screen. Accordingly, at least some portion of the bracket includes a flexible, transparent, polymeric material that allows the electronic screen to be viewed; and which allows operating room personnel to “touch” (not directly, of course, as this would compromise the integrity of the sterile field), and therefore manipulate, the touch-screen features and capabilities of the electronic device. Thus, in some versions of the invention, as shown in FIG. 9, the upper portion of the bracket 712 includes a flexible, transparent polymer or plastic 802 that allows operation of the electronic device's touch screen 804. In one version of the invention, the upper portion of the bracket includes a rigid frame around the perimeter of the upper portion, with the rigid frame attached to a flexible, transparent plastic that serves as a window through which the electronic device's screen is seen, and manipulated. The camera eye 806, on the underside of the enclosed electronic device, is positioned over the window 716 of the lower bracket portion.

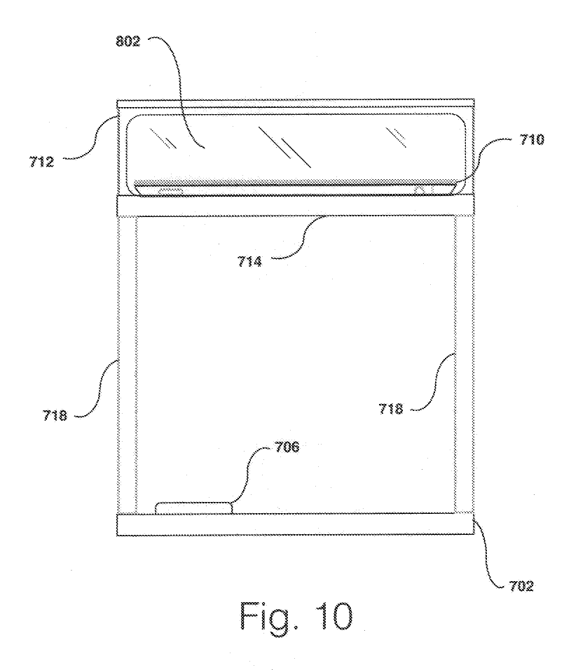

FIG. 10 shows a front view of one representative version of the landing pad, in this case a design incorporating a clam-shell-like bracket for receiving and enclosing an electronic device. The base 702 is attached to the upper bracket portion 712 and lower bracket portion 714 by a support 718 (as noted earlier, the support may be made up of separate pillars or columns; or a single piece; a person of skill will recognize that angled bracing members, or other conventional mechanical designs may be used to provide stability to the landing pad). The upper bracket portion 712 includes a transparent window 802 that allows the touch screen of the electronic device 710 to be seen and used. The lower bracket portion 714 includes an opening or transparent window that allows the electronic device 710 to take digital pictures of the suture cartridge 706 (or, alternatively, used sutures in a Styrofoam block; or other packaged, consumable items).

Other versions of the bracket are possible, so long as an electronic device enclosed therein, or supported thereon: (i) is isolated from contact with people or instruments within the sterile field (thus helping to maintain the integrity of the sterile field); (ii) is available to take digital pictures of sterile suture cartridges; (iii) allows viewers to see the electronic display; and (iv) allows users to manipulate the touchscreen capability of the device.

So, for example, another version of the bracket includes only a bottom portion. In order to isolate an electronic device from contact, the device is placed in a sterile, flexible, transparent bag of a size sufficient to enclose the electronic device. Once the device is placed inside the bag, the bag may be clamped using a sterile clamp or clamps (such as a hemostatic clamp); or otherwise sealed or closed. The enclosed device may then be placed on the lower portion of the bracket, with the electronic screen facing upward, and the camera eye facing downward. In order to help position the device, and reduce the chances of the device sliding off of the bottom portion of the bracket, the bottom portion may define a depression or recess within which the en-sleeved and clamped (or otherwise enclosed) electronic device rests. A person of skill will recognize that other conventional configurations are possible, so long as the electronic device is stably supported, with the device's touch screen available for viewing and use, and the device's camera eye having a clear line of sight for taking digital pictures.

In some versions of the disposable landing pad, a transparent material or film is adhered, attached, or placed on the electronic device. Alternatively, or in addition to, placement on the electronic device, the transparent material or film may be adhered, attached, or placed on at least some portion of the bottom portion of the bracket after the electronic device is placed thereon. Either way, the transparent material or film is then interposed between the electronic device (which is passed into the sterile field and placed in the bracket) and people and instruments inside the sterile field.

As noted elsewhere in the present application, a holder may be an integral part of the base (e.g., a feature that molded into the base itself). For example, the holder may be a depression or recess in the base that is of a size and shape capable of accepting the range of sterile suture cartridges that might be used. Alternatively, the holder may include a mechanical feature that stabilizes or positions a sterile suture cartridge (e.g., a feature akin to the clips or other such feature that stabilizes and positions a slide for viewing through a microscope). Because of cost, however, a holder, if present, is preferably molded into the base of the disposable landing pad.

For image-analysis purposes (or for suture-cartridge placement purposes—if, for example, the base does not include a holder), the landing pad may include one or more of the following disposed on the base: a circle; a series of concentric circles; one or more rays emanating from a central point; perpendicular, intersecting lines (as with a crosshair); or a grid of lines. Alternatively, or in addition to, such patterns disposed on the surface of the base, such reference lines or circles may be disposed electronically (e.g., via software program instructions) on the electronic image of a suture cartridge.

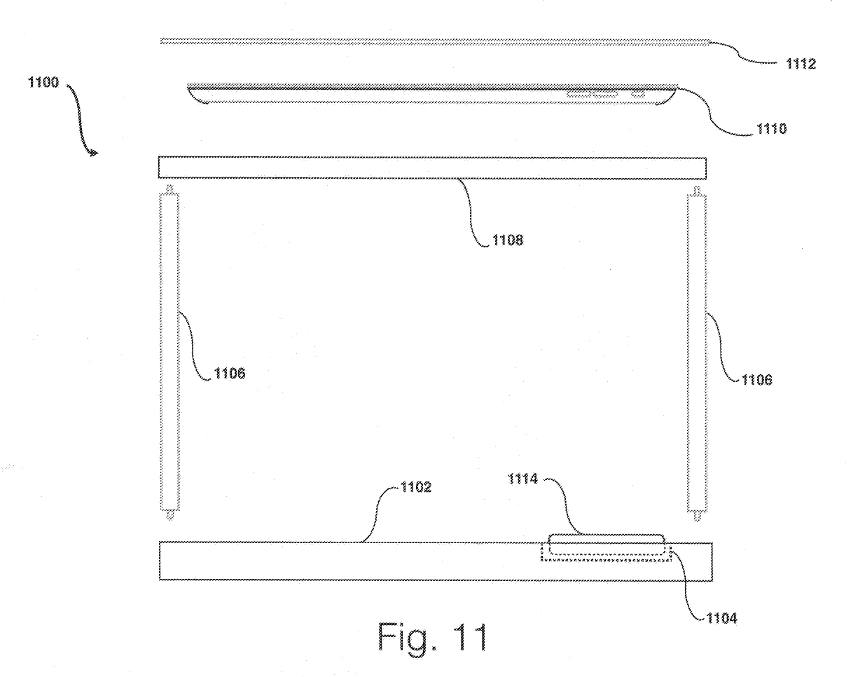

FIG. 11 shows a side view of another version of a landing pad 1100. In this embodiment of the invention, the landing pad 1100 comprises a base 1102 that includes a holder 1104. A support 1106—the support comprising a plurality of pillars—attaches the base 1102 to a bracket 1108. (Note: FIG. 11 shows the base, support, and bracket separate from one another, to show that the landing pad may be comprised of separate components that attach to one another. In this case the base and support include slots or holes (not shown), configured to receive pins or protrusions emanating from the end of the pillars making up the support. These pins or protrusions are inserted into the holes or slots when assembling the landing pad before use. A person of skill will recognize that other conventional configurations may be used to facilitate the attachment of different components to one another.) In this case the bracket 1108 is not a clamshell design, but instead is a tray-like design configured to receive and hold an electronic device 1110. The electronic device 1110, once placed in the bracket, is overlaid with a substantially transparent film/plastic layer 1112 (alternatively, as discussed elsewhere, the electronic device could be placed in a transparent bag, with the bag then sealed or clamped). The transparent layer (or bag) allows the electronic screen of the electronic device 1110 to be seen and used, and interposes a sterile material (the transparent layer of bag) between a user of the device and the device itself. The tray-like bracket 1108 includes a transparent portion (e.g., a transparent window) or opening that aligns with the downward facing camera eye, which is itself positioned over the holder 1104 and suture cartridge 1114. The transparent portion or opening, as well as the downward facing camera eye of the electronic device 1110, are not shown.

In preferred versions of the invention, the distance between the base 1102 and the bracket 1108 is selected to correspond to a focal length and field of view (not shown), that allows for imaging of the range of sterile suture cartridges to be placed in the holder 1104. Furthermore, the landing pad itself, in preferred versions of the invention, helps ensure that the distance between the camera eye and suture cartridge are substantially the same for each of the sterile suture cartridges that are imaged.

As discussed above, the landing pad may be a disposable article of manufacture. If the landing pad is disposable, then it would be packaged in a manner analogous to a sterile suture cartridge (i.e., the landing pad, or components of the landing pad, would be sterilized as part of the manufacturing and/or packaging process, with the landing pad or its components contained in a package having a non-sterile exterior surface, but which, when opened, allows a user access to the sterile contents of the package). Alternatively, the landing pad may be durable, and made of materials that allow for sterilization of the landing pad after each use. For example, many hospitals sterilize durable surgery items by heating the items to a specified temperature for a specified duration of time (i.e., the items are autoclaved). An exemplary condition for autoclaving includes subjecting equipment to high-pressure, saturated steam at a temperature of around 121 degrees Celsius for about 15 to 20 minutes or so. Thus, if the landing pad is durable, it is made of materials that allow the landing pad to be sterilized after each use in an operating room (such as by autoclaving the landing pad). Materials of construction that may be autoclaved include various polymers (e.g., polypropylene, polyphenyl-sulfone, high-density polyethylene, acetal co-polymer, some grades of Nylon and polycarbonate, etc.) and various metals or alloys. A person of skill will recognize that a variety of autoclavable materials may be used in constructing a durable landing pad of the present invention. Also, other methods may be used to sterilize medical equipment (e.g., methods deploying gamma radiation, ethylene oxide, or an electronic beam), but these alternatives are generally not preferred over autoclaving.

In some versions of the invention, an external camera is connected to an electronic device such as a smartphone, electronic tablet, desktop computer, portable computer, or other processing system. Any non-sterile component deployed within the sterile field of the operating room would typically be placed in flexible, transparent, sterile materials to prevent contamination. In these versions of the invention, the landing pad is configured to releasably engage, and position, the external camera in a desired spatial relationship to a sterile suture cartridge being imaged. Thus the base, as described above, is designed to provide stability to the landing pad as a whole. As before, the holder, if present, is configured to receive and position a sterile suture cartridge for imaging. Unlike the versions described above, the bracket is configured to receive and position the external camera—not the electronic device itself—in a desired spatial relationship to a sterile suture cartridge placed in the holder. A support then connects the bracket to the base. The external camera, the electronic device, and any connector between the device and camera would be placed in flexible, transparent, plastic bags or sleeves to help ensure that the integrity of the sterile field is preserved. In some versions of the invention, the electronic device remains outside the sterile field, but is connected to a camera deployed within the sterile field. In this case, the electronic device outside the sterile field need not be placed in a sterile, transparent plastic bag. But the camera, and any connector between the electronic device and camera, would be placed in sterile plastic bags.

A person of ordinary skill will recognize that the landing pad need not include a base, but such a configuration is not preferred. With no base, the background portion of a digital image of a suture cartridge is more likely to vary, depending on the surface on which the bracket and its support are placed (versus having a consistent background—the background provided by the surface of the base and any markings thereon—when the landing pad includes such a base). Furthermore, when compiling and populating the suture-cartridge database, choosing a background for the suture-cartridge digital images used in suture-cartridge database records that is the same or similar to the background for suture-cartridge digital images taken in the sterile field of an operating room is advantageous for image analysis (though not required). A landing pad that includes a base helps ensure that the background is the same or similar (if a landing pad is used both for helping compile and populate a suture-cartridge database, and for tallying in-count, out-count, and waste categories of sutures during surgery). Finally, in some versions of the present invention, systems and processes for suture (or other consumable item) tracking are deployed without a landing pad.

A person of ordinary skill will recognize that an electronic device used in conjunction with the landing pad is, in fact, a powerful computer/processing system. And this computer—whether it is a smartphone, an electronic tablet, a portable computer, a desktop computer, or other such device—must be configured to carry out certain steps necessary to the inventive method. First, as discussed above, the electronic device must be capable of taking digital pictures of the object being imaged, in this case a sterile suture cartridge (or a Styrofoam block on which are embedded used suture needles). The digital camera may either be a part of the electronic device itself, as with, for example, smartphones and electronic tablets. Or the digital camera may be external to, and connected with, the electronic device. Typically the camera comprises a charge-coupled device (“CCD”).

Second, the electronic device—alone or in conjunction with other computers/processing systems with which the electronic device communicates over a network—must be capable of executing the processes associated with comparing a computer-readable description of a digital image of a sterile suture cartridge taken in the sterile field of an operating room with a plurality of computer-readable descriptions of digital images associated with a plurality of suture-cartridge database records. Typically an image-analysis process is used to map a digital image into a computer-readable file or description that can be compared with the computer-readable files or descriptions of other digital images. In other words, the inventive method requires an image-recognition algorithm configured to search and correctly match the computer-readable description of the digital image of a sterile suture cartridge in the sterile field of an operating room to its corresponding database record. A number of image-analysis technologies and/or software may be used. For example, image-recognition software packages and/or technologies are available from companies such as Attrasoft, Inc., a business having offices in Savannah, Ga.; and Idee, Inc., a business having offices in Toronto, Ontario. Also, examples of image-recognition technologies are described, for example, in: U.S. Pat. No. 7,773,800 B2 to Ying Liu, entitled “Attrasoft Image Retrieval,” granting on Aug. 10, 2010 from U.S. application Ser. No. 10/078,299 (which is hereby incorporated by reference in its entirety, in a manner consistent herewith); U.S. Pat. No. 7,477,780 B2 to Wayne C. Boncyk and Ronald H. Cohen, entitled “Image Capture and Identification System and Process,” granting on Jan. 13, 2009 from U.S. application Ser. No. 10/492,243 (which is hereby incorporated by reference in its entirety, in a manner consistent herewith). Furthermore, optical-character recognition may be used to identify strings of text, numbers, or both (e.g., strings corresponding to the name of the manufacturer; name or model number; etc.). Once these strings are identified for a particular suture cartridge, then fields associated with the identified strings could be searched for matches. After a match was identified, a digital image of the sterile suture cartridge associated with the suture cartridge record could be displayed on screen of an electronic device, with operating room personnel then having an opportunity to confirm whether the suture-cartridge record identified as a match is correct. If the match is correct, then operating room personnel could confirm the match, thereby allowing the count of surgical sutures to increase by the surgical suture count associated with that particular sterile suture cartridge (with the count of surgical sutures associated with a sterile suture cartridge typically being one; but this count can and does vary). Note too that more than one algorithm may be used to identify a match between the digital picture of a sterile suture cartridge passed into the sterile field of an operating room with its corresponding suture-cartridge database record (e.g., image recognition and optical-character recognition). A person of skill will recognize that a number of algorithms may be adapted for use in matching the image of a suture cartridge present in a digital picture taken in the sterile field of an operating room, with a digital image retrieved from a plurality of such images contained in a suture-cartridge database.

### Additional Detail on Representative Versions of the Method and System

A digital image taken of a sterile suture cartridge within the sterile field of an operating room is used to search a suture-cartridge database that includes a plurality of suture-cartridge database records. Each record corresponds to a specific suture cartridge, with the record including a computer-readable file corresponding to a digital image of the suture cartridge. Image analysis is used to match the imaged sterile suture cartridge within the sterile field of an operating room with its corresponding suture-cartridge database record.

The method, then, presupposes the compiling of an image-searchable database of suture-cartridge database records. Each record corresponds to a unique suture-cartridge type from a specific manufacturer. Each suture-cartridge database record may include: the name of the manufacturer or supplier; the name and/or model number of the surgical suture; a digital image of a cross-sectional view of the needle; a digital image of a side view of the needle; a digital image of the sterile suture cartridge (note: the digital image of the sterile suture cartridge may be relied on to provide the cross-sectional and side views of the needle, given that the cartridge often includes these views of the surgical suture contained therein); numerical specifications of size and length (e.g., length of thread attached to needle; metric and/or non-metric gauge sizes of needle); textual descriptions of the surgical suture; materials of construction of needle and thread; method(s) of sterilization; etc. The suture-cartridge database record can also include a Universal Product Code for that surgical suture though, as discussed elsewhere, this code is typically not present on the sterile suture cartridge itself. By making a digital image of each and every sterile suture cartridge passed into the sterile operating field, and matching this digital image, obtained during surgery, to a specific suture-cartridge database record, the number (and type; as well as other characteristics) of incoming surgical sutures may be tracked and/or displayed.

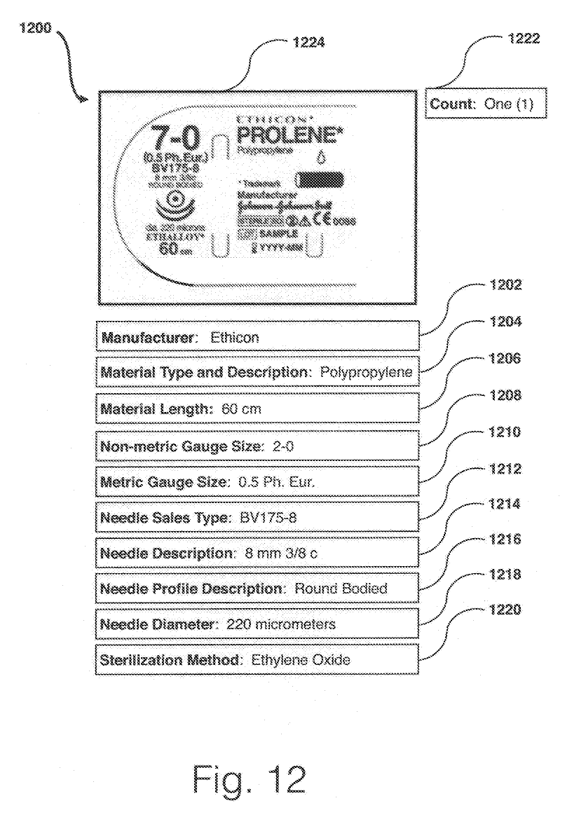

One example of a suture-cartridge database record 1200 is shown in FIG. 12. Of course FIG. 12 shows the record as it might be displayed, and does not show the computer-readable code representing the information and image as depicted in FIG. 12. The record itself comprises a number of fields characterizing suture characteristics that include, but are not limited to: the identity of the manufacturer 1202 (here, the field labeled “Manufacturer” includes the text “Ethicon”); the material type and description 1204 of the thread attached to the needle (here, the field labeled “Material Type and Description” includes the text “Polypropylene”); the length 1206 of the thread (here the field labeled “Material Length” includes the alpha-numeric string “60 cm,” meaning the thread length is 60 centimeters); the non-metric gauge size 1208 of the needle (here the field labeled “Non-Metric Gauge Size” includes the alpha-numeric string “2-0,” meaning the United States Pharmocopeia [“U.S.P.”] designation for a given suture size); the metric gauge size 1210 of the needle (here the field labeled “Metric Gauge Size” includes the alpha-numeric string “0.5 Ph. Eur.,” meaning the European Pharmocopeia [“Ph. Eur.”] designation for a given suture size); the manufacturer's sales type 1212 of the needle (or, for example, the product catalogue number; here the field labeled “Needle Sales Type” includes the alpha-numeric text “BV175-8”); the needle description 1214 (here the field labeled “Needle Description” includes the alpha-numeric text “8 mm ⅜ c,” meaning that the needle has an actual length of 8 millimeters, with this length defining three-eighths of a circle); the needle profile description 1216 (here the field labeled “Needle Profile Description” includes the text “Round Bodied,” meaning that the needle's cross-section is round); the needle diameter 1218 (here the field labeled “Needle Diameter” includes the alpha-numeric string “220 micrometers,” referring to the diameter of the needle); the sterilization method 1220 (here the field labeled “Sterilization Method” includes the text “Ethylene Oxide,” referring to the manufacturing method by which the surgical suture is sterilized during packaging); the count 1222 of individual surgical sutures contained within the sterile suture cartridge (here the field labeled “Count” includes the alpha-numeric text “One (1),” referring to the number of surgical sutures in the depicted sterile suture cartridge); and a digital image 1224 of a product label associated with the sterile suture cartridge. Of course a suture-cartridge database record may contain more or less information than that shown in the exemplary record depicted in FIG. 12. For example, the suture-cartridge database record might also include: an image of the non-sterile suture package used to contain the sterile suture cartridge until use; an image of the needle's profile, cross section, or both; an image of the bar code or other scannable label associated with that particular surgical suture (again, such labels typically are not disposed on the surface of the sterile suture cartridge; but typically are present on other packaging associated with the sterile suture cartridge, such as the non-sterile suture package); other such information or images useful to operating room personnel, inventory management professionals, or others concerned with optimizing and improving health-care services. Furthermore, the digital image of the suture cartridge itself may include only a printed label, or the entire suture cartridge.

Individual records of the suture-cartridge database may be created in various ways. For example, one version of the landing pad described above could be used to take digital pictures of each of the surgical sutures that are used at a given hospital. Each digital picture would then be associated with a unique database record for the surgical suture depicted in that particular picture. A template for each suture-cartridge database record could be designed to include fields for each piece of information deemed relevant by the users of the database. Alpha-numeric information or other data would then be entered, uploaded, or otherwise inputted into each of the fields. This information and data may be inputted in various ways including, for example, manual data entry; uploading information from files, spreadsheets, and the like; etc. As noted elsewhere, the information for the database may be compiled, and/or the database populated, by the user of the database or another party.

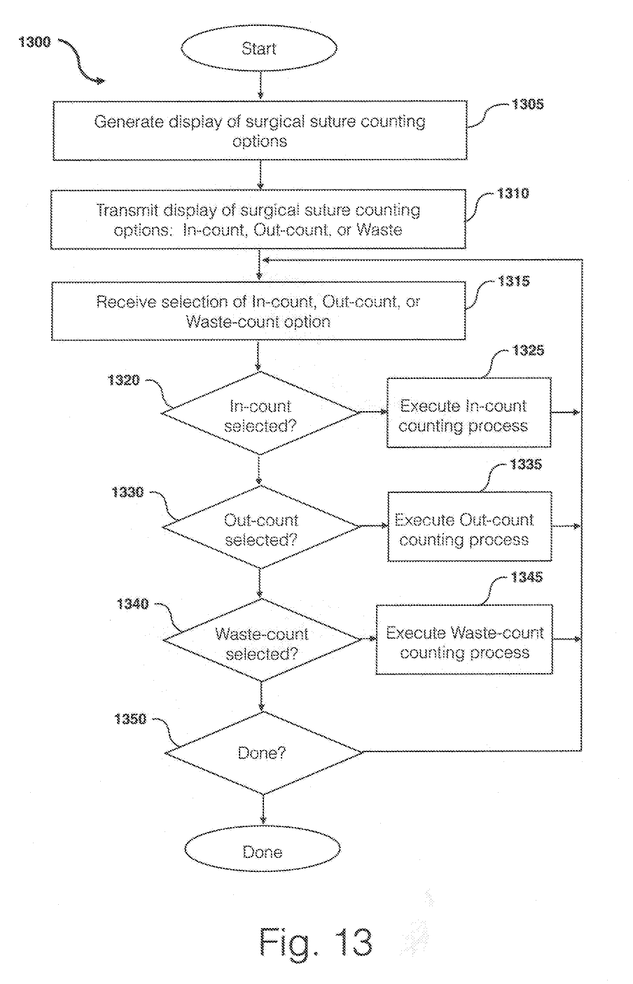

Steps 420, 425, 430, 435, and 440 of process 400, depicted in FIGS. 4 and 5 above, implicate other processes which are now described. As noted earlier, a user of the present invention is given options on how to count suture cartridges, and sutures, in the sterile field of an operating room. FIG. 13 shows one version of a process 1300 for providing an interface in which one of these options is selected.

First, in step 1305, a display is generated by the processing unit of the processing system executing process 1300. The display includes options for counting surgical sutures in the sterile field of an operating room. As noted elsewhere, these options may be denominated as the in-count option (for sterile suture cartridges passed into the sterile field of the operating room); the out-count option (for used sutures); and the waste option (for sterile suture cartridges that, at the end of the surgery, were not used, and therefore are wasted).

In step 1310, the display is transmitted. If a processing system is accessing process 1300 via a network connection (and if the accessing processing system is different from the processing system executing at least some of the steps of process 1300, such as steps 1305 and 1310), then the processing system executing at least some of the steps of process 1300 transmits the display to the electronic device (i.e., accessing process system) of the user. If the processing system accessing and executing process 1300 are one in the same, then the display is transmitted to the display device of processing system. One skilled in the art will recognize that different instructions may be needed to generate the display depending on which device receives the display.

As discussed above, FIG. 6 shows one version of a display that includes options for counting surgical sutures. In the representative version illustrated in FIG. 6, a user may use the touch screen of the electronic device to select, for example, the in-count counting option (i.e., “New Suture,” denominated as 608); the out-count counting option (i.e., the “Used/Styrofoam Pad,” denominated as 610); or the waste counting option (i.e., “Waste Suture,” denominated as 612).

Returning, then, to FIG. 13: in step 1315, the selection of a counting option is received. The option may be received as a request from a processing system or as an input into the processing system, depending on how the interface is being executed. For example, the request may be a “click” on a “button” of a screen (or the “touch” of an icon on a touch screen) if the processing system is directly performing the process; or a request message generated by an electronic device such as a laptop, desktop computer, workstation, electronic tablet, or smartphone in response to a click on a button (or the touch of an icon on a touch screen) on the display of the electronic device, if the electronic device is connected to the processing system executing process 1300.

In step 1320, process 1300 determines whether a request for an in-count was received. If an in-count counting option was received, then process 1300 performs step 1325 and then process 1300 returns to step 1315 to receive other counting options.

In step 1330, process 1300 determines whether a request for an out-count was received. If an out-count counting option was received, then process 1300 performs step 1335 and then process 1300 returns to step 1315 to receive other counting options.

In step 1340, process 1300 determines whether a request for a waste-count was received. If a waste-count counting option was received, then process 1300 performs step 1345 and then process 1300 returns to step 1315 to receive other counting options.

In step 1350, process 1300 determines whether a quit, exit, or end case command was received. If so, then process 1300 ends. Otherwise process 1300 returns to step 1315 to receive other counting options.

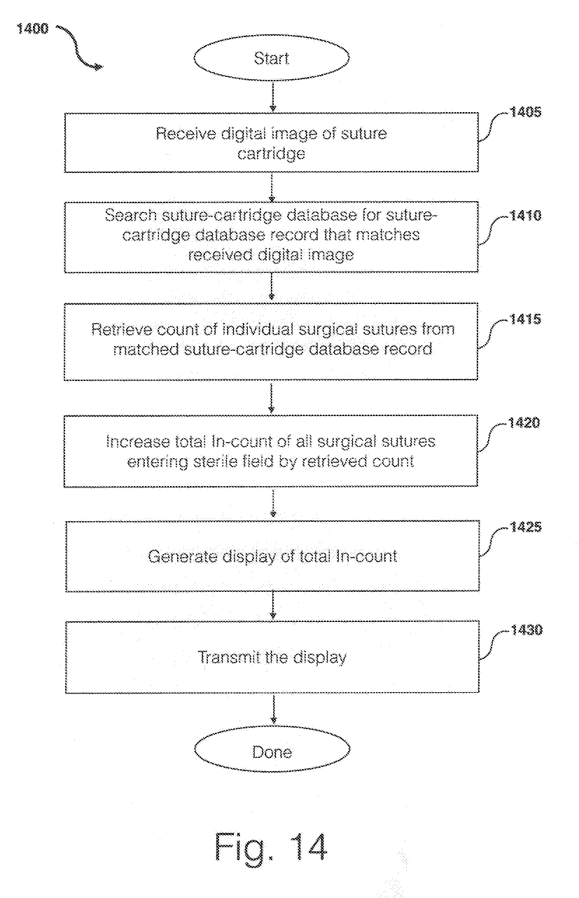

FIG. 14 illustrates one version of a process 1400 for counting new sterile suture cartridges entering the sterile field of an operating room. Process 1400 corresponds to step 1325 of process 1300 illustrated in FIG. 13.

In step 1405, a computer-readable file corresponding to the digital image of a suture cartridge is received. In step 1410, process 1400 deploys an image-recognition algorithm, examples of which are referenced elsewhere in the present application, to match the computer-readable file of the digitally imaged suture cartridge with a suture-cartridge database record. As discussed earlier, each suture-cartridge database record includes a computer-readable file of a digital image of the specific suture cartridge characterized by that record. Once the matching suture-cartridge database record is identified, the number characterizing the count of individual sutures contained in the imaged suture cartridge is retrieved, as illustrated in step 1415. The total in-count of all surgical sutures entering the sterile field is then increased by the retrieved count, as shown in step 1420. A display that includes the updated in-count is then generated and transmitted, as shown in steps 1425 and 1430. Process 1400 then ends.

It should be noted that process 1400 emphasizes the real-time tallying of the total number of sutures entering the sterile field, primarily for safety purposes. As is explained elsewhere, however, other information may be retrieved from matched suture-cartridge database records for subsequent generation of a suture-use analytics database, in which suture use and waste is correlated with other surgery variables.

The physical context for the execution of process 1400 is typically as follows. A person outside the sterile field opens a non-sterile suture package that contains the sterile suture cartridge contained therein. For example, if the non-sterile suture package comprises a peelable foil top, then the person outside the sterile field removes the foil without contacting the sterile suture cartridge contained therein. The person outside the sterile field then “flips” the sterile suture cartridge onto a sterile surface within the sterile field (e.g., a sterile tray). Operating room personnel within the sterile field then take a digital picture of the sterile suture cartridge using an electronic device/processing system as described above.

In one version of the invention, this digital image is taken using a version of the sterile, disposable landing pad described generally above. In this case, process 1400 is preceded by a person outside the sterile field opening a non-sterile package containing the sterile landing pad contained therein. For example, in some versions of the invention, the sterile landing pad—or unassembled components of the sterile landing pad—would be “flipped” onto a sterile surface within the sterile field (e.g., a sterile tray or table). Once flipped into the sterile field, operating room personnel within the sterile field would unfold, assemble, or otherwise fully deploy the landing pad so that it was ready for use. After the landing pad was deployed, a person outside the sterile field would then pass an electronic device into the sterile field. If, for example, the electronic device is a tablet having both a touch screen and a camera, then the electronic device might be carefully placed in a sterile plastic bag (with the person outside the sterile field not touching the plastic bag; and with personnel inside the sterile field not touching the electronic device being dropped or inserted into the plastic bag). After the electronic device was placed in the bag, operating room personnel would then clamp or seal the plastic bag. The electronic device would then be available for use in conjunction with the landing pad (e.g., a version of the landing pad in which the electronic device, en-sleeved by the sterile plastic bag, is placed on a bracket of the landing pad).

In other versions of the landing pad, operating room personnel outside the sterile field would deposit the electronic device directly into the bracket of the landing pad (e.g., into an opened, clam-shell-like bracket). Again, the person outside the sterile field would be careful not to touch the landing pad-especially those surfaces of the landing pad outside the interior of the clam-shell-like bracket-when depositing the electronic device into the interior of the bracket. And operating room personnel within the sterile field would be careful to avoid touching both the electronic device and the interior of the clam-shell-like bracket. After the electronic device was deposited in the lower portion of the open, clam-shell-like bracket, operating room personnel within the sterile field would then close the bracket (contacting only the sterile exterior of the bracket during its closure).

In other versions of the present invention, the landing pad comprises a bracket lacking an upper portion. Here, as above, an electronic device would be deposited in the bracket. Then operating-room personnel within the sterile field of the operating room would apply a sterile transparent plastic layer or film to the electronic device, bracket perimeter, or both—with the transparent plastic layer or film serving to preserve the integrity of the sterile field (with the layer or film interposed between the electronic device and operating-room personnel within the sterile field of the operating room). A person of skill will recognize that other combinations of steps in which an electronic device is deployed for imaging within the sterile field while, at the same time, maintaining the integrity of the sterile field, are possible. Also, as described elsewhere in the present application, some versions of the landing pad are durable and autoclavable. Ifa durable landing pad is used, then, after autoclaving, the sterilized landing pad would be deployed in the sterile field of the operating room. An electronic device (e.g., an electronic tablet) would be placed in or on the sterilized landing pad generally as described above.

Process 1400 is typically repeated a plurality of times before processes 1500 and 1600 (other suture/suture-cartridge counting options) are carried out. For example, in preparing for surgery, operating personnel will typically deploy, within the sterile field, all of the equipment, tools, and materials anticipated as being needed by the surgeon(s) for that specific surgery. Process 1400, then, would be repeated for each new suture cartridge passed into the sterile field of an operating room before a surgery begins.

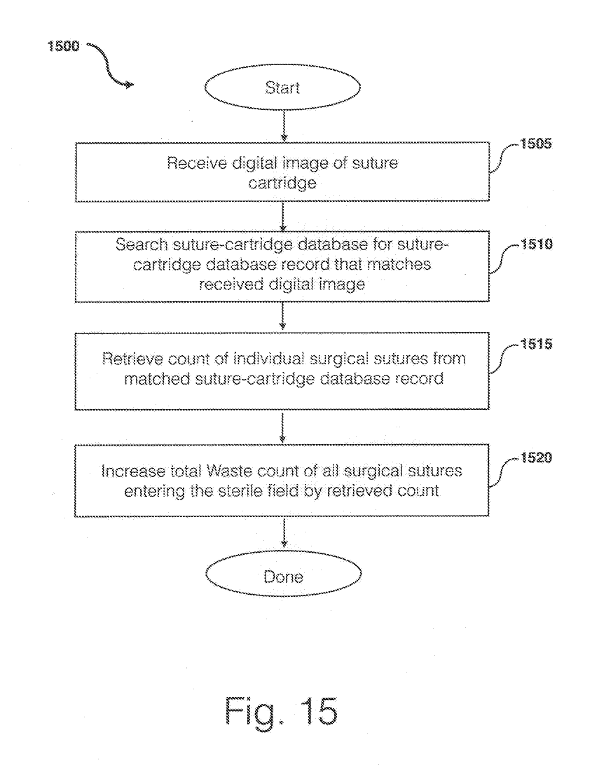

FIG. 15 illustrates one representative version of a process 1500 in which the total number of wasted sutures is determined. In step 1505, a computer-readable file corresponding to the digital image of a suture cartridge is received. In step 1510, process 1500 deploys an image-recognition algorithm, examples of which are referenced elsewhere in the present application, to match the computer-readable file of the digitally imaged suture cartridge with a suture-cartridge database record. As discussed earlier, each suture-cartridge database record includes a computer-readable file of a digital image of the specific suture cartridge characterized by that record. Once the matching suture-cartridge database record is identified, the number characterizing the count of individual sutures contained in the imaged suture cartridge is retrieved, as illustrated in step 1515. The total waste-count of all surgical sutures entering the sterile field, but not used, is then increased by the retrieved count, as shown in step 1520. While not shown in FIG. 15, if desired, a display that includes the updated waste count may then be generated and transmitted. Process 1500 then ends.

It should be noted that, rather than search the entire suture-cartridge database for a matching suture-cartridge database record, as shown in 1510, process 1500 can be modified to search only those suture-cartridge database records that were matched to new sterile suture cartridges entering the sterile field of the operating room. Also, as with the representative process 1400 illustrated in FIG. 14, process 1500 may retrieve other information from matched suture-cartridge database records for subsequent generation of a suture-use analytics database, in which suture use and waste are correlated with other surgery variables.

The physical context for the execution of process 1500 is typically as follows. As discussed above, once the non-sterile suture package containing the sterile suture cartridge is opened—with the sterile suture cartridge being flipped into the sterile field of the operating room—the sterile suture cartridge must be used for that surgery or it is wasted. Thus, typically at the end of a surgery, those sterile suture cartridges that have not been used (and which still contain non-deployed surgical sutures) are counted using process 1500. As with process 1400, the general steps are: (1) the step of taking a digital picture of each sterile suture cartridge passed into the sterile field of the operating room, but not used during the surgery; (2) matching the digital picture of the unused sterile suture cartridge with its corresponding suture-cartridge database record (using one or more image-recognition, optical-character recognition, or other such algorithms, as discussed above); and (3) increasing the “Waste” count of unused surgical sutures by the number of individual surgical sutures associated with that particular unused sterile suture cartridge, as indicated by the suture-cartridge database field corresponding to the count of individual surgical sutures in that particular suture cartridge.

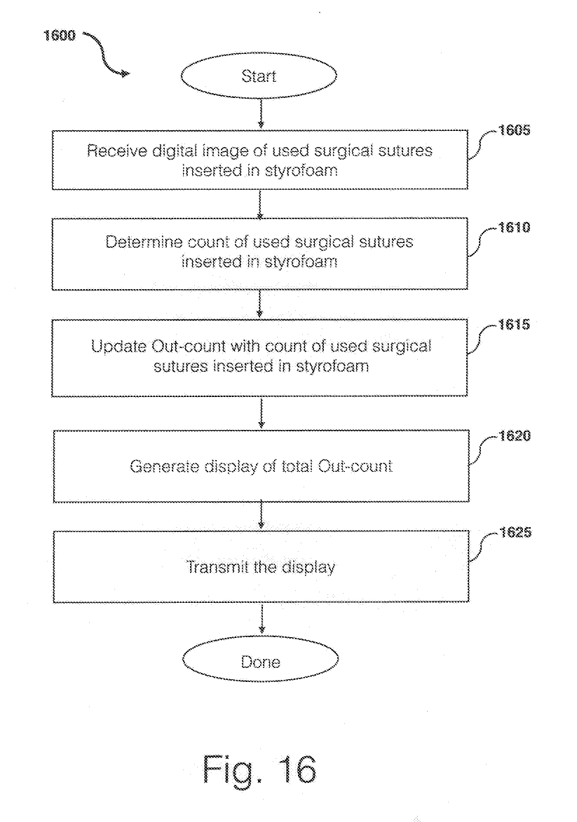

FIG. 16 illustrates one representative version of a process 1600 for counting used surgical sutures. In step 1605, a computer-readable file corresponding to the digital image of used sutures embedded in a Styrofoam block is received. In step 1610, process 1600 deploys an image-analysis algorithm to determine the total number of sutures embedded in the Styrofoam block. The total out-count of all surgical sutures entering the sterile field, and used, is then updated with the then-current count, as shown in step 1615. A display that includes the updated out-count is then generated and transmitted, as shown in steps 1620 and 1625. Process 1600 then ends.

The physical context for the execution of process 1600 is typically as follows. First, it should again be noted that the manner in which used surgical sutures are tracked is different from the processes and methods used to track sterile suture cartridges entering the sterile field; and those suture cartridges inside the sterile field that are not used by the time the surgery ends. After a surgical suture is used—that is, after a surgeon has passed the needle and attached thread through tissue multiple times, thereby joining the tissue—the used needle and unused thread is severed from the thread joining the tissue. The used needle is then segregated from unused surgical sutures by, for example, inserting the used needle into a Styrofoam block. A used needle is not disposed of because, as discussed above, used and unused surgical sutures must be carefully tracked to ensure that a needle is not inadvertently left in a patient.

In the present invention, the same electronic device used to take digital pictures of sterile suture cartridges is also used to take digital pictures of used needles. In the case of used needles, however, the different types of surgical sutures (i.e., needles) are typically not tracked directly. Instead, only the cumulative number of used needles is tracked directly. For example, if used needles are placed side-by-side on a white Styrofoam block—with the sharp end of the needle inserted into the Styrofoam block, and with some space between each individual used needle—then a digital picture of that block presents a series of substantially dark, spaced-apart lines or regions, with each dark line or region corresponding to an individual needle. Operating room personnel wishing to count the number of used needles at a particular point, or points, during the surgery, would take a digital picture of the used needles inserted into the Styrofoam block (after selecting the appropriate counting option). Image analysis (e.g., binary/bimodal image analysis of the image, with black representing objects—in this case suture needles; and white representing the background—the Styrofoam block; or vice versa [suture needles represented by white; Styrofoam block represented by black]) would then be used to determine the count of needles embedded in the Styrofoam block. The tally of used needles, denoted as the “Out Count” in process 1500, would be replaced by the current total of used needles inserted into the Styrofoam block. It should be noted, though, that other materials and methods may be used for segregating, and securing for counting, surgical sutures that are actually used during surgery. For example, used needles may be attached to a substrate having an adhesive disposed on the surface of the substrate, thereby helping ensure that used needles are not lost, and which may be counted using image analysis.

It should be noted that processes 1400, 1500, and 1600 need not be completed in sequence. For example, at or before the beginning of an operation, a number of sterile suture cartridges may be passed into the sterile field of the operating room and counted using process 1400. Generally, process 1400 will be repeated for each sterile suture cartridge passed into the sterile field before any used surgical sutures are counted by process 1600; and before the determination of any wasted sutures by process 1500. Later, during the surgery, the number of used sutures might be determined by process 1600. If the surgeon, or surgeons, needed more or different surgical sutures than anticipated, then operating room personnel would again return to counting the additional sterile suture cartridges being passed into the sterile field of the operating room (i.e., using process 1400). Typically the number of wasted surgical sutures is not determined until the end of the surgery. It should be noted, though, that one or more of these processes might be performed at different times before, during, and at the end of a surgical procedure.

### Additional Detail on Tracking Suture Use During Surgery; and Compiling and Populating a Suture-Use Analytics Database

The previous paragraphs describe the processes and steps by which the “In Count,” “Out Count,” and “Waste” count are tracked for a particular surgery. Additional detail is provided in the paragraphs below.

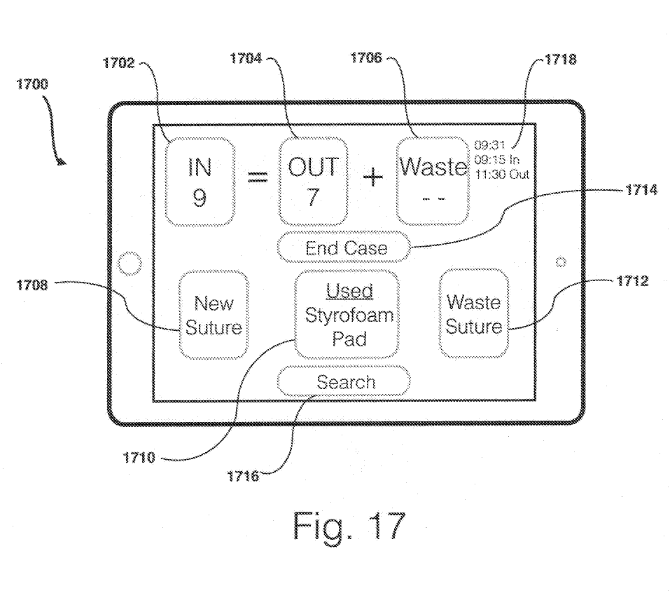

During a given surgery, the surgeon(s) and other operating room personnel are concerned with the safety and health of the patient. Accordingly, these personnel are primarily focused on ensuring that a needle is not inadvertently left inside the patient. As a result, the inventive method can include steps in which the “In Count,” “Out Count,” and “Waste”—or some combination thereof—are displayed during the course of the surgery. Another representative example of such a display is presented in FIG. 17. Here the electronic device 1700 includes an electronic screen that functions both as a display, and as a touch screen for manipulating or inputting information. This version of a display generally shows an equation in which the “In” count 1702 of surgical sutures equals the “Out” count 1704 of used surgical sutures plus the “Waste” count 1706 of unused surgical sutures. Other ways of displaying these counts are possible. For example, as shown in FIG. 6, a display might not include a counter for the wasted sterile suture cartridges. Instead, at the conclusion of a surgery, the wasted suture cartridges could be counted using process 1500, and a calculation then performed by the electronic device/processing system to verify that the Waste count equaled the difference between the In Count and the Out Count. The electronic device might then be configured to display a symbol or text to confirm that all surgical sutures passing into the sterile field are properly accounted for.

The version of a display in FIG. 17 also includes various touch-pad “buttons” or icons: a “New Suture” button 1708; a “Used/Styrofoam Pad” button 1710; and a “Waste Suture” button 1712. Operating room personnel wishing to count a sterile suture cartridge passing into the sterile field would first “push” (i.e., touch the button icon, indirectly, by placing a gloved finger or thumb on the transparent, flexible, plastic interposed between the electronic screen and the user) the “New Suture” button 1708 to activate the electronic device's camera, and to account for the imaged surgical suture (or sutures, if the sterile suture cartridge included more than one individual surgical suture) under the “In” counter of the display (i.e., to execute process 1400, which corresponds to the selected in-count counting option). The process can be configured so that operating room personnel must push the appropriate touch-pad button for each sterile suture cartridge being imaged, and accounted for. It should be noted that both the number, and type, of each sterile suture cartridge passing into the sterile field of the operating room is tracked. Operating room personnel, during a surgery, are primarily interested in accounting for all surgical sutures—to ensure that no needle is inadvertently left in the patient. Nevertheless, there may be times, during a surgery, that operating room personnel are interested in viewing more detailed information about specific types of surgical sutures that have been passed into the sterile field (e.g., shape, size, materials of construction, etc.). Thus the inventive method and system encompasses generating tabular or graphic reports listing or correlating one or more pieces of information regarding the surgical sutures that have been passed into the sterile field during a particular surgery. Also, as is discussed below and elsewhere in the application, this same information can be aggregated over time for a plurality of surgeries, resulting in a suture-use analytics database that can be searched and analyzed in various ways.

During a typical surgery, which can last several hours, operating room personnel can periodically update the total number of used surgical sutures by touching the “Used/Styrofoam Pad” button 1710. A user touching button 1710 activates process 1600 in which the electronic device's camera is activated, with the resulting digital picture analyzed for the total number of needles inserted into, for example, a Styrofoam pad. The resulting number is not added to the current “Out” count, but instead replaces the current “Out” count. Operating room personnel, by viewing the electronic device's display screen, can compare the “In” count of not-yet-used surgical sutures to the then-current “Out” count of used surgical sutures. Any difference between the “In” count and “Out” count should equal the number of surgical sutures contained in the not-yet-used sterile suture cartridges within the sterile field.

At the end of a surgery, any not-yet-used sterile suture cartridge within the sterile field is then accounted for as waste. A user touches the “Waste Suture” button 1712—or other such icon, touch-screen image, or text corresponding to unused surgical sutures in the sterile field—in order to count the wasted sutures. As before, touching button 1712 configures the electronic device for taking a digital picture; and, in accordance with process 1500, processing the computer-readable file corresponding to the digital picture so that the imaged sterile suture cartridge is matched with its corresponding suture-cartridge database record, and accounted for as waste.

The representative version of a display shown in FIG. 17 includes two additional touch-screen buttons. Once a surgery is over, and all surgical sutures deployed in the sterile field are accounted for, a user touches the “End Case” button 1714 to signify that the collected data is complete for that particular surgery. If the duration of the surgery is being tracked, then touching the “End Case” button 1714 will also stop any timer that was started at the beginning of the surgery. In effect, touching button 1714 signifies that a data set for a particular surgery is to be compiled and available for analysis. The data set for a particular case can be analyzed by itself, or, as is more likely, combined with or compared to other data sets (e.g., data sets for other surgeries of the same type, whether at the same hospital, or different hospitals; data sets for surgeries of the same type, but performed by different surgeons; etc.).

One representative version of a suture-use analytics database is analogous to a spreadsheet that includes a plurality of columns and rows. Each column specifies a suture-use characteristic or operating-room variable; and each row specifies a record corresponding to a completed surgery. So, for example, column headers (with each header representing a variable or constant for which information is inputted) might include (in no particular order): surgery date; surgery type; surgery duration; surgery start time; surgery end time; surgeon name(s); operating room personnel names; number of shifts required during surgery; operating room identifier; hospital identifier; suture #1 model number; suture #1 manufacturer; suture #1 used; suture #1 waste; calculated cost of suture #1 waste; suture #2 model number . . . [etc., for each suture type used during a given surgery]; etc. Persons of skill will recognize that other database formats are possible. The individual fields of the database could be populated automatically upon completion of a surgery when, for example, operating room personnel press the “End Case” button 1714 in FIG. 17. Over time, a hospital (or multiple hospitals, if data is shared across the hospitals) can build a comprehensive suture-use analytics database that provides for graphs, reports, and other data summaries correlating suture use and waste with other suture characteristics and operating variables.

The representative version of a display shown in FIG. 17 also includes a “Search” button 1716. During a surgery, or at some other time (e.g., when preparing for a surgery), a user, by depressing button 1716, may search data collected using the inventive apparatus, method, and system. As discussed elsewhere in the application, the inventive landing pad, method, and system are used to track the number and type of used, and wasted, surgical sutures for a particular surgery. Furthermore, the inventive method provides for inputting and/or tracking other operating-room variables that may then be correlated with the number and type of used and wasted surgical sutures. These operating room variables, as described elsewhere in the present application, might include: surgeon name; surgery type; surgery duration; surgery start time; names of operating room personnel; one or more characteristics of the patient; number of shift changes for a particularly long surgery; identity of operating room (e.g., operating room number); identity of hospital in which the surgery is performed; identity of the hospital or health-care system in which the surgery is performed; etc. By aggregating such data over time, health-care personnel may search the database for a variety of purposes. For example, a nurse or other person preparing supplies and equipment for a surgery can search the database for the identity of the number and type of surgical sutures used by a particular surgeon for a particular surgery. Alternatively, hospital administrators may search the database to determine whether the number of wasted surgical sutures is related, in a statistically significant manner, to some other operating room variable, such as suture type; surgery type; surgeon identity; etc. In other words, the collected data may be used in myriad ways that help the health-care community become better, and more efficient, at providing safe and effective health-care services.

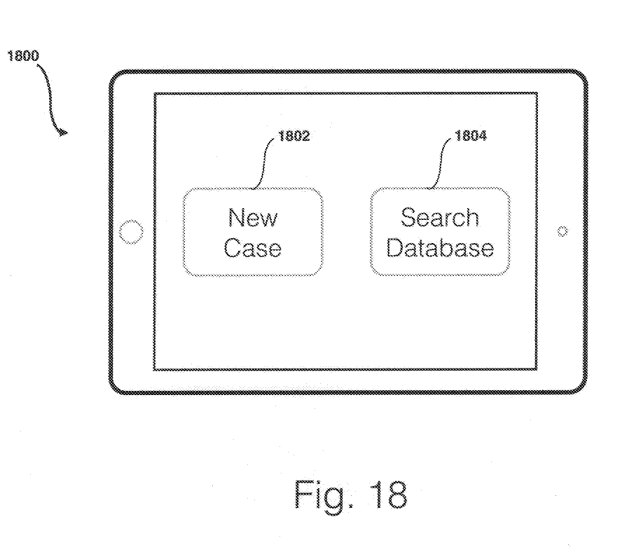

Of course the advantage of an electronic device having a touch screen for a display is that it can be configured in many different ways for manipulating and displaying information. FIG. 18 shows a screen in which two options are presented to a user of the invention. Such a screen may be viewed as that available prior to a surgery beginning—i.e., an entry screen or pre-surgery screen. Thus, for example, and as mentioned above, a nurse or other health-care professional might first search a database correlating surgical suture characteristics to other operating-room variables (e.g., a surgeon's past selection and use of surgical sutures for a particular surgery). The nurse might use this information, for example, to obtain the numbers and types of surgical sutures used by that surgeon in the past for an upcoming surgery. For this purpose, the nurse or other user touches the “Search Database” button 1804, which then would configure the electronic device and display such that the nurse could search the suture-use analytics database for correlations between variables of interest (e.g., selecting or entering a surgeon's name, and selecting or entering a surgery type, would result in the suture-use analytics database being searched for records that included both the surgeon's name, and the surgery type, and then displaying the variable(s) of interest, such as the number and type of surgical sutures used by this particular surgeon in the past for the specified surgery type).

A user would touch the “New Case” button 1802 at the start of a surgery. By touching button 1802, the electronic device would be configured to display a screen like that depicted in FIGS. 6 or 16; or, in accordance with process 300, a display facilitating the inputting of operating-room input variables. Furthermore, the time at which button 1802 was depressed could be recorded, with the duration of the surgery equaling the difference in time between when button 1802 was depressed, and when the “End Case” button 1714 of FIG. 17 is touched.

Different representative versions of screens, icons, text, or other images are presented the present application. A person of skill will recognize that an electronic device/processing system may be configured to display other versions of such screens, icons, text, or other images without departing from the inventive method and system for tracking the use and waste of surgical sutures.

Also, the preceding paragraphs disclose an inventive apparatus, method, and system used to track a consumable item deployed during surgery (in this case surgical sutures). The term “consumable” means that the item, once passed into the sterile field, is used or, if not used, disposed of. It should be recognized that the aforementioned method, apparatus, and/or system may be used with other consumable items deployed during surgery, whether or not these packaged items display a UPC code. I.e., the same method, system, and/or apparatus may be readily adapted to encompass tracking the use and waste of other packaged consumable items passed into the sterile field of an operating room, and correlating such use and waste with other operating-room variables.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

We claim:

1. A landing pad comprising:
a base configured to receive and position a sterile suture cartridge;
a bracket configured to receive and position an electronic device that includes a camera and a touch screen, the bracket including an opening configured to align with the electronic device's camera so that the camera has a clear line of sight to the sterile suture cartridge;
a support having opposing ends, wherein one end of the support is attached to the base, and wherein the other end of the support is attached to the bracket;
wherein the combination of the base, bracket, and support position the electronic device and sterile suture cartridge in a spaced-apart relationship for digital imaging of the sterile suture cartridge by the electronic device.

2. The landing pad of claim 1 wherein the bracket comprises an upper portion and a lower portion.

3. The landing pad of claim 2 wherein the upper portion and lower portion are attached by a hinge.

4. The landing pad of claim 2 wherein the upper portion of the bracket comprises a transparent portion through which the touch screen is viewed.

5. The landing pad of claim 1 further comprising a package containing the base, bracket, and support, wherein the base, bracket, and support are sterile.

6. The landing pad of claim 5 wherein the base, bracket, and support are separate parts configured to attach to one another.

7. The landing pad of claim 1 wherein the landing pad is autoclavable.

8. A method of tracking the number of surgical sutures used during a surgery, the method comprising the steps of:
(a) receiving a digital picture of a sterile suture cartridge passed into the sterile field of an operating room, wherein the sterile suture cartridge includes one or more surgical sutures;
(b) matching the digital picture with a suture-cartridge database record, the suture-cartridge database record comprising a plurality of suture characteristics, wherein at least one of the suture characteristics is the count of the number of surgical sutures in the digitally imaged sterile suture cartridge;
(c) retrieving the count of the number of surgical sutures in the sterile suture cartridge from the matched suture-cartridge database record;
(d) increasing a sum of the number of surgical sutures passed into the sterile field of an operating room by the retrieved count; and
(e) displaying the sum of the number of surgical sutures passed into the sterile field of the operating room.

9. The method of claim 8 further comprising the steps of:
(a) receiving a digital picture of used surgical sutures;
(b) determining the total number of used surgical sutures from the digital picture of used surgical sutures; and
(c) displaying the total number of used surgical sutures.

10. The method of claim 8 further comprising the steps of:
(a) receiving a digital picture of a sterile suture cartridge passed into the sterile field of an operating room, but not used, wherein the sterile suture cartridge includes one or more surgical sutures;
(b) matching the digital picture of the not-used suture cartridge with a suture-cartridge database record, the suture-cartridge database record comprising a plurality of suture characteristics, wherein at least one of the suture characteristics is the count of the number of surgical sutures in the digitally imaged sterile suture cartridge; and
(c) retrieving the count of the number of surgical sutures in the not-used suture cartridge from the matched suture-cartridge database record;
(d) increasing a sum of the number of not-used surgical sutures by the retrieved count.

11. The method of claim 10 wherein the suture-cartridge database record for the not-used suture cartridge includes the name of the manufacturer of the not-used suture cartridge and the manufacturer's suture model number of the not-used suture cartridge, and wherein a sum of the number of not-used surgical sutures is determined for the manufacturer's suture model number.

12. The method of claim 11 further comprising the steps of:
receiving an operating-room variable; and
correlating the operating-room variable with the sum of not-used surgical sutures for each manufacturer's suture model number.

13. A tangible computer-readable storage medium having instructions stored thereon, execution of which, by a processing system, causes the processing system to perform operations comprising:
receiving a digital image of a suture cartridge; and
matching the received digital image of a suture cartridge to a suture-cartridge database record, the suture-cartridge database record comprising a plurality of suture-cartridge database fields, wherein one of the suture-cartridge database fields is the count of individual sutures contained in the suture cartridge.

14. The tangible computer-readable storage medium of claim 13, the operations further comprising retrieving the count of individual sutures from the suture-cartridge database record.

15. The tangible computer-readable storage medium of claim 14, the operations further comprising:
receiving an option to count the digitally imaged suture cartridge as a new suture cartridge passing into a sterile field of an operating room;
increasing the total number of new sutures entering the sterile field of the operating room by the retrieved count.

16. The tangible computer-readable storage medium of claim 15, wherein the suture-cartridge database record includes the name of a manufacturer of the new suture cartridge and the manufacturer's suture model number of the new suture cartridge, and wherein the total number of new surgical sutures entering the sterile field of an operating room is determined for each manufacturer's suture model number.

17. The tangible computer-readable storage medium of claim 16, the operations further comprising:
receiving an operating-room variable; and
correlating the operating-room variable with the total number of new surgical sutures entering the sterile field of the operating room for each manufacturer's suture model number.

18. The tangible computer-readable storage medium of claim 14, the operations further comprising:
receiving an option to count the digitally imaged suture cartridge as a suture cartridge passed into the sterile field of an operating room, but not used;
increasing the total number of not-used sutures by the retrieved count.

19. The tangible computer-readable storage medium of claim 18, wherein the suture-cartridge database record includes the name of the manufacturer of the not-used suture cartridge and the manufacturer's suture model number of the not-used suture cartridge, and wherein the total number of not-used surgical sutures is determined for each manufacturer's suture model number.

20. The tangible computer-readable storage medium of claim 19, the operations further comprising:
receiving an operating-room variable; and
correlating the operating-room variable with the total number of not-used surgical sutures for each manufacturer's suture model number.
